(19) United States
(12) Patent Application Publication
(10) Pub. No.: US 2016/0324962 A1
(43) Pub. Date: Nov. 10, 2016

(54) COMPOSITIONS AND METHODS FOR TREATING SARCOMA

(71) Applicant: MEDIMMUNE, LLC, Gaithersburg, MD (US)

(72) Inventor: HAIHONG ZHONG, GAITHERSBURG, MD (US)

(21) Appl. No.: 15/105,954

(22) PCT Filed: Dec. 17, 2014
(86) PCT No.: PCT/US14/70862

§ 371 Date: Jun. 17, 2016

Related U.S. Application Data
(60) Provisional application No. 61/918,227, filed on Dec. 19, 2013.

(51) Int. Cl. A61K 39/395; A61K 31/5377; A61K 9/00; A61K 31/436

(52) U.S. Cl. CPC A61K 39/3955; A61K 31/436; A61K 31/5377; A61K 9/0019; A61K 9/0053; A61K 2039/542

(57) ABSTRACT

The present invention provides compositions and methods for the treatment of sarcoma. The compositions comprise an antibody that binds at least one of IGF-1 and IGF-2 and an mTOR inhibitor. The mTOR inhibitor may be AZD2014 or rapamycin.

### SEQUENCE LISTING

The instant application contains a Sequence Listing which has been submitted electronically in ASCII format and is hereby incorporated by reference in its entirety. Said ASCII copy, created on Dec. 16, 2014, is named IGF-110WO1_SL.txt and is 9,921 bytes in size.

### BACKGROUND OF THE INVENTION

Sarcomas are neoplasias from transformed cells of mesenchymal origin, including osteosarcoma and soft tissue sarcoma. Soft tissue sarcomas are the fifth most common solid tumour in children under 20 years old, with rhabdomyosarcoma being the most common type. Osteosarcomas are the third most common cancer in adolescence, with the two most common types being osteosarcoma and Ewing's sarcoma. Sarcomas also affect adults but at lower frequency.

Sarcomas exhibit a wide variety of histologic types and can occur anywhere in the body. At present, treatment options are surgery, with adjuvant radiation used selectively for high-grade, incompletely resected lesions. Chemotherapy has been shown to be of limited benefit, delaying time to recurrence but not affecting overall survival.

Advances in the combined use of chemotherapy, surgery, and radiation have improved the survival of rhabdomyosarcoma patients with localized disease. Between 1975 and 2002, the 5-year survival rate has increased from 53% to 65% for children younger than 15 years and from 30% to 47% for adolescents aged 15 to 19 years. However, in rhabdomyosarcoma patients metastatic disease remains a major predictor of poor outcome, and has not been significantly impacted by combination therapy.

For osteosarcoma patients, present treatment options include surgery and chemotherapy for micrometastatic disease, which is present but not detectable in most patients at diagnosis. Although radiotherapy is an important treatment for soft tissue sarcoma, osteosarcomas are uniformly resistant to radiation. While cure rates for localized osteosarcoma using combination therapies are in the range of 60-70%, patients who present with metastases or multifocal disease have a poor prognosis. With long-term survival rates of less than 25%, osteosarcoma has one of the lowest survival rates for pediatric cancer.

Therefore, compositions and methods for reducing the proliferation and survival of sarcoma cells, and for treating sarcoma are urgently required.

### SUMMARY OF THE INVENTION

As described below, the present invention features compositions and methods for the treatment of sarcoma, particularly proliferating tumor cells (e.g., induced by IGF-1/-2) within the sarcoma. The compositions comprise an mTOR inhibitor and an antibody that specifically binds to at least one of IGF-1 and IGF-2.

In an embodiment, the invention refers to a pharmaceutical composition for the treatment of sarcoma comprising an effective amount of an mTOR inhibitor and an effective amount of an antibody that specifically binds to at least one of insulin-like growth factor 1 (IGF-1) and insulin-like growth factor 2 (IGF-2). In some embodiments the antibody in the pharmaceutical composition neutralizes a least one of IGF-1 and IGF-2.

In particular embodiments of the invention, the antibody in the pharmaceutical composition comprises a heavy chain complementarity determining region 1 (CDR1) comprising the amino acid sequence set forth in SEQ ID NO: 1 (Ser Tyr Asp Ile Asn); a heavy chain complementarity determining region 2 (CDR2) comprising the amino acid sequence set forth in SEQ ID NO: 2 (Trp Met Asn Pro Asn Ser Gly Asn Thr Gly Tyr Ala Gln Lys Phe Gln Gly); a heavy chain complementarity determining region 3 (CDR3) comprising the amino acid sequence set forth in SEQ ID NO: 3 (Asp Pro Tyr Tyr Tyr Tyr Tyr Gly Met Asp Val); a light chain complementarity determining region 1 (CDR1) comprising the amino acid sequence set forth in SEQ ID NO: 4 (Ser Gly Ser Ser Ser Asn Ile Glu Asn Asn His Val Ser); a light chain complementarity determining region 2 (CDR2) comprising the amino acid sequence set forth in SEQ ID NO: 5 (Asp Asn Asn Lys Arg Pro Ser); and a light chain complementarity determining region 3 (CDR3) comprising the amino acid sequence set forth in SEQ ID NO: 6 (Glu Thr Trp Asp Thr Ser Leu Ser Ala Gly Arg Val).

In some embodiments, the antibody in the pharmaceutical composition of the invention comprises one or more variable regions comprising an amino acid sequence selected from the amino acid sequences set forth in SEQ ID NO: 7 and SEQ ID NO: 8. In particular embodiments, the antibody in the pharmaceutical composition of the invention has the amino acid sequence of the antibody produced by hybridoma cell line 7.159.2 (ATCC Accession Number PTA-7424).

In some embodiments, the pharmaceutical composition of the invention comprises an mTOR inhibitor selected from the group consisting of AZD2014, INK128, AZD8055, NVP-BEZ235, BGT226, SF1126, PKI-587, rapamycin, temsirolimus, everolimus, ridaforolimus, and combinations thereof. In particular embodiments, the mTOR inhibitor in the pharmaceutical composition of the invention comprises rapamycin. In particular embodiments, the mTOR inhibitor in the pharmaceutical composition of the invention comprises AZD2014.

In some embodiments, the pharmaceutical composition of the invention is used to treat a sarcoma selected from the group consisting of Ewing's sarcoma, Osteosarcoma, Rhabdomyosarcoma, Askin's tumor, Sarcoma botryoides, Chondrosarcoma, Malignant Hemangioendothelioma, Malignant Schwannoma, soft tissue sarcoma, Alveolar soft part sarcoma, Angiosarcoma, Cystosarcoma Phyllodes, Dermatofibrosarcoma protuberans, Desmoid Tumor, Desmoplastic small round cell tumor, Epithelioid Sarcoma, Extraskeletal chondrosarcoma, Extraskeletal osteosarcoma, Fibrosarcoma, Hemangiopericytoma, Hemangiosarcoma, Kaposi's sarcoma, Leiomyosarcoma, Liposarcoma, Lymphangiosarcoma, Lymphosarcoma, Malignant peripheral nerve sheath tumor, Neurofibrosarcoma, Synovial sarcoma, and Undifferentiated pleomorphic sarcoma.

In an embodiment, the invention refers to a method for reducing the survival or proliferation of a sarcoma cell. The method comprises contacting at least one sarcoma cell with a pharmaceutical composition comprising an mTOR inhibitor and an antibody that specifically binds at least one of IGF-1 and IGF-2; measuring the survival or proliferation of the sarcoma cell contacted with the pharmaceutical composition and the survival or proliferation of a sarcoma cell not contacted with the pharmaceutical composition; comparing the survival or proliferation of the sarcoma cell contacted with the pharmaceutical composition with the survival or proliferation of the sarcoma cell not contacted with the pharmaceutical composition; wherein the survival or proliferation of the sarcoma cell treated with the pharmaceutical composition is reduced as compared with the survival or proliferation of the sarcoma cell not treated with the pharmaceutical composition.

In an embodiment, the invention relates to a method for treating sarcoma in a subject comprising administering to the subject a pharmaceutical composition comprising an mTOR inhibitor and an antibody that specifically binds at least one of IGF-1 and IGF-2. In particular embodiments of the invention, the antibody that specifically binds at least one of IGF-1 and IGF-2 neutralizes at least one of IGF-1 and IGF-2.

In particular embodiments, the antibody used in the method for treating sarcoma comprises a heavy chain complementarity determining region 1 (CDR1) comprising the amino acid sequence set forth in SEQ ID NO: 1 (Ser Tyr Asp Ile Asn); a heavy chain complementarity determining region 2 (CDR2) comprising the amino acid sequence set forth in SEQ ID NO: 2 (Trp Met Asn Pro Asn Ser Gly Asn Thr Gly Tyr Ala Gln Lys Phe Gln Gly); a heavy chain complementarity determining region 3 (CDR3) comprising the amino acid sequence set forth in SEQ ID NO: 3 (Asp Pro Tyr Tyr Tyr Tyr Tyr Gly Met Asp Val); a light chain complementarity determining region 1 (CDR1) comprising the amino acid sequence set forth in SEQ ID NO: 4 (Ser Gly Ser Ser Ser Asn Ile Glu Asn Asn His Val Ser); a light chain complementarity determining region 2 (CDR2) comprising the amino acid sequence set forth in SEQ ID NO: 5 (Asp Asn Asn Lys Arg Pro Ser); and a light chain complementarity determining region 3 (CDR3) comprising the amino acid sequence set forth in SEQ ID NO: 6 (Glu Thr Trp Asp Thr Ser Leu Ser Ala Gly Arg Val). In particular embodiments of the invention, the antibody that specifically binds at least one of IGF-1 and IGF-2 comprises one or more variable regions comprising the amino acid sequence selected from the amino acid sequences set forth in SEQ ID NO: 7 and SEQ ID NO: 8.

In particular embodiments, the mTOR inhibitor used in the method for treating sarcoma is at least one of AZD2014, INK128, AZD8055, NVP-BEZ235, BGT226, SF1126, PKI-587, rapamycin, temsirolimus, everolimus, and ridaforolimus.

In particular embodiments, the sarcoma treated by the methods of the invention is one of more of Ewing's sarcoma, Osteosarcoma, Rhabdomyosarcoma, Askin's tumor, Sarcoma botryoides, Chondrosarcoma, Malignant Hemangioendothelioma, Malignant Schwannoma, soft tissue sarcoma, Alveolar soft part sarcoma, Angiosarcoma, Cystosarcoma Phyllodes, Dermatofibrosarcoma protuberans, Desmoid Tumor, Desmoplastic small round cell tumor, Epithelioid Sarcoma, Extraskeletal chondrosarcoma, Extraskeletal osteosarcoma, Fibrosarcoma, Hemangiopericytoma, Hemangiosarcoma, Kaposi's sarcoma, Leiomyosarcoma, Liposarcoma, Lymphangiosarcoma, Lymphosarcoma, Malignant peripheral nerve sheath tumor, Neurofibrosarcoma, Synovial sarcoma, and Undifferentiated pleomorphic sarcoma.

In particular embodiments of the invention, the pharmaceutical composition is administered at 10 mg/kg, 30 mg/kg, or 60 mg/kg. In some embodiments, the method of treating sarcoma of the invention inhibits tumor growth in the subject by at least about 10%, 25%, 50%, 75% or more relative to a reference. In particular embodiments, the method of treating sarcoma of the invention inhibits sarcoma cell proliferation.

In particular embodiments, the pharmaceutical compositions of the invention are administered by intravenous injection or oral administration. In particular embodiments, in the methods of treatment of the invention, the antibody and the mTOR inhibitor are administered concurrently, within about 1 hour to about 24 hours, or within about 1 day to about 3 days.

In an embodiment, the invention refers to a method for treating a subject having Ewing's sarcoma, osteosarcoma, or rhabdomyosarcoma. In a particular embodiment, the method comprises administering to the subject an effective amount of MEDI-573 and rapamycin. In a particular embodiment, the method comprises administering to the subject an effective amount of MEDI-573 and AZD2014.

In an embodiment, the invention relates to a kit for treating sarcoma. The kit comprises an effective amount of an mTOR inhibitor and an antibody that specifically binds IGF-1 and/or IGF-2, and instructions for using the kit to treat sarcoma. In a particular embodiment of the invention, the kit comprises MEDI-573 antibody and rapamycin. In a particular embodiment of the invention, the kit comprises MEDI-573 antibody and AZD2014.

### BRIEF DESCRIPTION OF THE DRAWINGS

FIG. 1A to FIG. 1D—Depict the calculated ΔCt for IGF-1, IGF-2, IGF-1R, and the IRA:IRB ratio calculated using the mRNA levels detected by quantitative reverse transcription polymerase chain reaction (qRT-PCR) in primary tumor xenografts from pediatric sarcomas. FIG. 1A depicts the calculated ΔCt for IGF-1; FIG. 1B depicts the calculated ΔCt for IGF-2; FIG. 1C depicts the calculated ΔCt for IGF-1R; FIG. 1D depicts the calculated ΔCt IR-A:IR-B ratio.

FIG. 2A and FIG. 2B—Depict the calculated ΔCt for IGF-1, IGF-2, IGF-1R, and the IRA:IRB ratio calculated using the mRNA levels detected by qRT-PCR in sarcoma cell lines. FIG. 2A depicts the calculated ΔCt for IGF-1, IGF-1R, IGF-2, and IGF2R. FIG. 2B depicts the calculated ΔCt for IR-A:IR-B ratios.

FIG. 3A to FIG. 3C—Depict the of IGF-1, IGF-2, and IGF-1R protein levels detected in sarcoma cell lines using ELISA. FIG. 3A depicts the levels of IGF-1; FIG. 3B depicts the levels of IGF-2; and FIG. 3C depicts the levels of IGF-1R.

FIG. 4A to FIG. 4F—Depict the effect of MEDI-573 on the cell viability in autocrine driven Sarcoma Cell lines. FIG. 4A depicts the cell viability of RD-ES cells; FIG. 4B depicts cell viability of TC-71 cells; FIG. 4C depicts cell viability of SJCRH30 cells; FIG. 4D depicts cell viability of SK-ES-1 cells; FIG. 4E depicts cell viability of SJS1 cells; FIG. 4F depicts cell viability of RD cells.

FIG. 5A to FIG. 5F—Depict the effect of MEDI-573 treatment on the Growth and Proliferation of IGF-Induced Ewing's sarcoma cell lines. FIG. 5A depicts cell viability of IGF-1-stimulated RD-ES cells; FIG. 5B depicts cell viability of IGF-2-stimulated RD-ES cells; FIG. 5C depicts cell viability of IGF-1-stimulated SK-ES-1 cells; FIG. 5D depicts cell viability of IGF-2-stimulated SK-ES-1 cells; FIG. 5E depicts cell viability of IGF-1-stimulated TC-71 cells; FIG. 5F depicts cell viability of IGF-2-stimulated TC-71 cells.

FIG. 6A to FIG. 6D—Depict the effect of MEDI-573 treatment on the Growth and Proliferation of IGF-Induced Osteosarcoma cell lines. FIG. 6A depicts cell viability of IGF-1 stimulated SAOS2 cells; FIG. 6B depicts cell viability of IGF-2 stimulated SAOS2 cells; FIG. 6C depicts cell viability of IGF-1 stimulated MG-63 cells; FIG. 6D depicts cell viability of IGF-2 stimulated MG-63 cells.

FIG. 7A to FIG. 7C—Depict the efficacy of MEDI-573 in sarcoma xenograft models with autocrine IGF-1 and IGF-2 signaling. FIG. 7A depicts tumor volume in RD-ES cells; FIG. 7B depicts the tumor volume in SJSA-1 cells; FIG. 7C depicts the tumor volume in KHOS/NP cells.

FIG. 8A to FIG. 8C—Depict the effect of adding different amounts of MEDI-573 to sarcoma xenograft models with hIGF-1 or hIGF-2 induced signaling. FIG. 8A depicts the hIGF-1 levels in RD-ES cells; FIG. 8B depicts the hIGF-2 levels in SJSA-1 cells; FIG. 8C depicts the hIGF-2 levels in KHOS/NP cells.

FIG. 9A to FIG. 9C—Depict the effect of the addition of MEDI-573 on the autophosphorylation of IGF-1R, IR-A, and Akt in RD-ES, SK-ES-1, TC-71, and KHOS cells. In each graph, the first bar represents the results from the untreated control; the second bar represents the results from adding the isotype control to the culture; and the third bar represents the results of treating the cells with MEDI-573. FIG. 9A depicts the levels of pIGF-1R; FIG. 9B depicts the levels of p1R-A; FIG. 9C depicts the levels of pAKT.

FIG. 10A to FIG. 10C—Depict the effect of the addition of MEDI-573 on IGF-1 and/or IGF-2 induced signalling in vitro. FIG. 10A depicts the levels of pIGF-1R; FIG. 10B depicts the levels of p1R-A; FIG. 10C depicts the levels of pAKT.

FIG. 11—Depicts an immunoblot showing the phosphorylation levels of pAKT and phosphorylated Eukaryotic translation initiation factor 4E-binding protein 1 (p4EBP1) obtained from tissues of mice bearing ˜400 mm³ RD-ES tumors. Left three lanes, no MEDI-573 added; right three lanes, MEDI-573 added.

FIG. 12A to FIG. 12D—Depicts graphs showing the levels of hIGF-1 and hIGF-2 in RD-ES tumor and plasma before and after treatment with MEDI-573.

FIG. 13—Depicts an immunoblot showing phosphorylation levels of pAKT, p4EBP1, and pS6K in untreated mice, in mice after induction with IGF-1, in mice after induction with IGF-2, in mice after induction with IGF-1 and treatment with MEDI-573, and in mice after induction with IGF-2 and treatment with MEDI-573. Samples from three different mice are shown in each group.

FIG. 14—Depicts the growth and proliferation of RD-ES cells treated with MEDI-573 and an mTOR inhibitor (rapamycin or AZD2014) alone or in combination with each other.

FIG. 15—Depicts an immunoblot showing phosphorylation levels of pAKT, p4EBP1, and pS6K in untreated cells, cells treated with MEDI-573 alone, cells treated with rapamycin alone, cells treated with rapamycin in combination with MEDI-573, cells treated with AZD2014 alone, and cells treated with MEDI-573 in combination with AZD2014.

FIG. 16A to FIG. 16B—Depict the growth and proliferation of sarcoma cells in RD-ES tumor xenografts treated with AZD2014, MEDI-573, AZD2014 in combination with MEDI-573 and controls. FIG. 16A growth and proliferation of cells; FIG. 16B body weight of mice treated.

FIG. 17A to FIG. 17B—Depict the growth and proliferation of sarcoma cells in RD-ES tumor xenografts treated with rapamycin, MEDI-573, rapamycin in combination with MEDI-573 and controls. FIG. 17A growth and proliferation of cells; FIG. 17B body weight of mice treated.

### BRIEF DESCRIPTION OF THE SEQUENCE LISTING

SEQ ID NO: 1 depicts the amino acid sequence of the MEDI-573 heavy chain complementarity determining region 1 (Ser Tyr Asp Ile Asn).

SEQ ID NO: 2 depicts the amino acid sequence of the MEDI-573 heavy chain complementarity determining region 2 (Trp Met Asn Pro Asn Ser Gly Asn Thr Gly Tyr Ala Gln Lys Phe Gln Gly).

SEQ ID NO: 3 depicts the amino acid sequence of the MEDI-573 heavy chain complementarity determining region 3 (Asp Pro Tyr Tyr Tyr Tyr Tyr Gly Met Asp Val).

SEQ ID NO: 4 depicts the amino acid sequence of the MEDI-573 light chain complementarity determining region 1 (Ser Gly Ser Ser Ser Asn Ile Glu Asn Asn His Val Ser).

SEQ ID NO: 5 depicts the amino acid sequence of the MEDI-573 light chain complementarity determining region 2 (Asp Asn Asn Lys Arg Pro Ser).

SEQ ID NO: 6 depicts the amino acid sequence of the MEDI-573 light chain complementarity determining region 3 (Glu Thr Trp Asp Thr Ser Leu Ser Ala Gly Arg Val).

SEQ ID NO: 7 depicts the amino acid sequence of the MEDI-573 variable heavy chain polypeptide:

Gln Val Gln Leu Val Gln Ser Gly Ala Glu Val Lys
Lys Pro Gly Ala Ser Val Lys Val Ser Cys Lys Ala
Ser Gly Tyr Thr Phe Thr Ser Tyr Asp Ile Asn Trp
Val Arg Gln Ala Thr Gly Gln Gly Leu Glu Trp Met
Gly Trp Met Asn Pro Asn Ser Gly Asn Thr Gly Tyr
Ala Gln Lys Phe Gln Gly Arg Val Thr Met Thr Arg
Asn Thr Ser Ile Ser Thr Ala Tyr Met Glu Leu Ser
Ser Leu Arg Ser Glu Asp Thr Ala Val Tyr Tyr Cys
Ala Arg Asp Pro Tyr Tyr Tyr Tyr Tyr Gly Met Asp
Val Trp Gly Gln Gly Thr Thr Val Thr Val Ser Ser
Ala

SEQ ID NO: 8 depicts the amino acid sequence of the MEDI-573 variable light chain polypeptide:

Gln Ser Val Leu Thr Gln Pro Pro Ser Val Ser Ala
Ala Pro Gly Gln Lys Val Thr Ile Ser Cys Ser Gly
Ser Ser Ser Asn Ile Glu Asn Asn His Val Ser Trp
Tyr Gln Gln Leu Pro Gly Thr Ala Pro Lys Leu Leu
Ile Tyr Asp Asn Asn Lys Arg Pro Ser Gly Ile Pro
Asp Arg Phe Ser Gly Ser Lys Ser Gly Thr Ser Ala
Thr Leu Gly Ile Thr Gly Leu Gln Thr Gly Asp Glu
Ala Asp Tyr Tyr Cys Glu Thr Trp Asp Thr Ser Leu
Ser Ala Gly Arg Val Phe Gly Gly Gly Thr Lys Leu
Thr Val Leu Gly

SEQ ID NO: 9 depicts the amino acid sequence of the MEDI-573 light chain polypeptide:

Gln Ser Val Leu Thr Gln Pro Pro Ser Val Ser Ala
Ala Pro Gly Gln Lys Val Thr Ile Ser Cys Ser Gly
Ser Ser Ser Asn Ile Glu Asn Asn His Val Ser Trp
Tyr Gln Gln Leu Pro Gly Thr Ala Pro Lys Leu Leu
Ile Tyr Asp Asn Asn Lys Arg Pro Ser Gly Ile Pro
Asp Arg Phe Ser Gly Ser Lys Ser Gly Thr Ser Ala
Thr Leu Gly Ile Thr Gly Leu Gln Thr Gly Asp Glu
Ala Asp Tyr Tyr Cys Glu Thr Trp Asp Thr Ser Leu
Ser Ala Gly Arg Val Phe Gly Gly Gly Thr Lys Leu
Thr Val Leu Gly Gln Pro Lys Ala Ala Pro Ser Val
Thr Leu Phe Pro Pro Ser Ser Glu Glu Leu Gln Ala
Asn Lys Ala Thr Leu Val Cys Leu Ile Ser Asp Phe
Tyr Pro Gly Ala Val Thr Val Ala Trp Lys Ala Asp
Ser Ser Pro Val Lys Ala Gly Val Glu Thr Thr Thr
Pro Ser Lys Gln Ser Asn Asn Lys Tyr Ala Ala Ser
Ser Tyr Leu Ser Leu Thr Pro Glu Gln Trp Lys Ser
His Arg Ser Tyr Ser Cys Gln Val Thr His Glu Gly
Ser Thr Val Glu Lys Thr Val Ala Pro Thr Glu Cys
Ser

SEQ ID NO: 10 depicts the amino acid sequence of the MEDI-573 heavy chain polypeptide:

Gln Val Gln Leu Val Gln Ser Gly Ala Glu Val Lys
Lys Pro Gly Ala Ser Val Lys Val Ser
Cys Lys Ala Ser Gly Tyr Thr Phe Thr Ser Tyr Asp
Ile Asn Trp Val Arg Gln Ala Thr Gly Gln
Gly Leu Glu Trp Met Gly Trp Met Asn Pro Asn Ser
Gly Asn Thr Gly Tyr Ala Gln Lys Phe Gln
Gly Arg Val Thr Met Thr Arg Asn Thr Ser Ile Ser
Thr Ala Tyr Met Glu Leu Ser Ser Leu Arg Ser
Glu Asp Thr Ala Val Tyr Tyr Cys Ala Arg Asp Pro
Tyr Tyr Tyr Tyr Tyr Gly Met Asp Val Trp
Gly Gln Gly Thr Thr Val Thr Val Ser Ser Ala Ser
Thr Lys Gly Pro Ser Val Phe Pro Leu Ala Pro
Cys Ser Arg Ser Thr Ser Glu Ser Thr Ala Ala Leu
Gly Cys Leu Val Lys Asp Tyr Phe Pro Glu
Pro Val Thr Val Ser Trp Asn Ser Gly Ala Leu Thr
Ser Gly Val His Thr Phe Pro Ala Val Leu Gln
Ser Ser Gly Leu Tyr Ser Leu Ser Ser Val Val Thr
Val Pro Ser Ser Asn Phe Gly Thr Gln Thr Tyr
Thr Cys Asn Val Asp His Lys Pro Ser Asn Thr Lys
Val Asp Lys Thr Val Glu Arg Lys Cys Cys
Val Glu Cys Pro Pro Cys Pro Ala Pro Pro Val Ala
Gly Pro Ser Val Phe Leu Phe Pro Pro Lys Pro
Lys Asp Thr Leu Met Ile Ser Arg Thr Pro Glu Val
Thr Cys Val Val Val Asp Val Ser His Glu
Asp Pro Glu Val Gln Phe Asn Trp Tyr Val Asp Gly
Val Glu Val His Asn Ala Lys Thr Lys Pro
Arg Glu Glu Gln Phe Asn Ser Thr Phe Arg Val Val
Ser Val Leu Thr Val Val His Gln Asp Trp
Leu Asn Gly Lys Glu Tyr Lys Cys Lys Val Ser Asn
Lys Gly Leu Pro Ala Pro Ile Glu Lys Thr Ile
Ser Lys Thr Lys Gly Gln Pro Arg Glu Pro Gln Val
Tyr Thr Leu Pro Pro Ser Arg Glu Glu Met
Thr Lys Asn Gln Val Ser Leu Thr Cys Leu Val Lys
Gly Phe Tyr Pro Ser Asp Ile Ala Val Glu
Trp Glu Ser Asn Gly Gln Pro Glu Asn Asn Tyr Lys
Thr Thr Pro Pro Met Leu Asp Ser Asp Gly
Ser Phe Phe Leu Tyr Ser Lys Leu Thr Val Asp Lys
Ser Arg Trp Gln Gln Gly Asn Val Phe Ser
Cys Ser Val Met His Glu Ala Leu His Asn His Tyr
Thr Gln Lys Ser Leu Ser Leu Ser Pro Gly
Lys

### DETAILED DESCRIPTION OF THE INVENTION

The invention features pharmaceutical compositions and methods that are useful for the treatment and prevention of sarcomas. The pharmaceutical composition for the treatment of sarcoma of the invention comprises an effective amount of an mTOR inhibitor and an effective amount of an antibody that specifically binds to at least one of insulin-like growth factor 1 (IGF-1) and insulin-like growth factor 2 (IGF-2). In some embodiments the antibody in the pharmaceutical composition neutralizes a least one of IGF-1 and IGF-2. The invention further provides compositions and methods for monitoring a patient having a sarcoma.

The present invention is based, at least in part, on the discovery that an antibody that neutralizes IGF-1 and/or IGF-2 when in combination with mTOR inhibitors (e.g., AZD2014, rapamycin) is useful for decreasing the proliferation, survival and/or increasing cell death of IGF-responsive sarcoma cells, including cells that secrete IGF-1 and/or IGF-2 in an autocrine manner.

MEDI-573 is a fully human monoclonal antibody that binds to IGF-2 with cross reactivity to IGF-1. MEDI-573 neutralizes IGF-1 and IGF-2 and inhibits signaling through both the IGF-1R and IR-A pathways. A hybridoma cell line (7.159.2) expressing MEDI-573 was deposited at the American Type Culture Collection (ATCC) on Mar. 7, 2006 and received the Patent Deposit Designation No. PTA-7424. A description of this antibody and its preparation is found in U.S. Pat. No. 7,939,637, issued May 10, 2011, which is hereby incorporated by reference in its entirety.

As described elsewhere, most sarcoma cell lines express IGF-1R and IGF-1, but only osteosarcoma cell lines and a few rhabdosarcoma cell lines secrete IGF-2. MEDI-573 inhibits in vitro proliferation of a number of sarcoma cell lines, with Ewing's sarcoma cell lines being most sensitive. The data presented here indicates that sarcoma cells respond to autocrine or paracrine growth stimulation by secreted IGF-1 and IGF-2. In addition, MEDI-573 inhibited IGF-1- and IGF-2-induced growth of sarcoma cells and significantly blocked IGF-1- and IGF-2-induced activation of the IGF-1R and AKT pathways. Growth inhibition of sarcoma xenografts by MEDI-573 was correlated with neutralization of IGF-1 and IGF-2 ligands.

As described here, MEDI-573 also inhibited rapamycin-induced AKT activation. A combination of MEDI-573 and mTOR inhibitor resulted in significantly enhanced anti-tumor activities in vivo. In summary, the data indicate that inhibiting IGF-1 and IGF-2 by MEDI-573 in combination with mTOR inhibitors (rapamycin or AZD2014) resulted in potent anti-tumor activity for various sarcomas. Advantageously, it has been found that targeting IGF-1 and/or IGF-2 is useful for treating sarcoma in combination with mTOR inhibitor, in contrast to targeting IGF receptors which has the potential to perturb insulin function. Accordingly, the invention provides pharmaceutical compositions and methods that are useful in treating subjects as having or having a propensity to develop a sarcoma, to develop a recurrence of sarcoma, and/or to develop metastatic sarcoma. In particular, the pharmaceutical compositions of the invention are useful for treating Ewing's sarcoma and some rhabdomyosarcoma.

### Insulin-Like Growth Factors (IGF)—IGF-1 and IGF-2

Insulin-like growth factors, IGF-1 and IGF-2, are growth factors involved in regulating cell proliferation, survival, differentiation, and transformation. Both ligands are expressed ubiquitously and act as endocrine, paracrine, and autocrine growth factors (Pollak, Nat Rev Cancer. 2008, 8(12):915-28; DeMeyts, BioEssays 2004, 26(12): 1351-1362, 2004; Tao et al., 2007, Nat Clin Pract Oncol. 4(10):591-602; Ryan and Goss, Oncologist. 2008, 13(1):16-24). Insulin-like growth factor-I and IGF-2 exert their various actions through binding to the insulin-like growth factor 1 receptor (IGF-1R) and insulin receptor A isoform (IR-A), activating multiple intracellular signaling cascades including the IRS proteins, Akt, and MAPK pathways (Sciacca et al., Oncogene. 1999, 18(15):2471-9; Chitnis et al. Clin Cancer Res. 2008, 14(20):6364-70; Belfiore et al., Endocr. Rev. 2009, 30, 586-623; Baserga, Future Oncol. 2009, 5(1):43-50). Receptors for IGF ligands include IGF receptors type 1 and type 2 (IGF-1R and IGF-2R), insulin receptors A and B (IR-A and IR-B), and hybrid receptors (IGF-1R/IR-A and IGF-1R/IR-B). IGF-2R preferentially binds IGF-2. However, IGF-2R lacks an intracellular kinase domain and does not mediate cell signaling. Without being bound to a particularly theory, loss of IGF-2R results in increased tumorigenicity, presumably by increasing the availability of IGF-2 to bind to IGF-1R. Both IGF-1 and IGF-2 exist as complexes in the circulatory system, bound to one of six IGF binding proteins (IGFBP-1 to IGFBP-6). IGFBP-3, in conjunction with a third molecule, acid labile subunit, forms a complex that accounts for the majority of circulating IGF. IGFBPs have a higher affinity for IGF than their cognate receptors and have the potential to sequester IGF from the receptor. However, alternative models indicate that the binding proteins may potentiate IGF activity, either by extending its half-life in circulation or by binding to certain molecules on the cell surface, thus providing a reservoir of available IGF to the cell.

High levels of circulating IGF-1 and -2 are associated with an increased risk for development of several common cancers (Renehan et al., Lancet. 2004, 363(9418):1346-53), including breast, prostate, pancreatic and colorectal cancer, non-small cell lung cancer (NSCLC), hepatocellular carcinoma (HCC), and sarcoma. The overexpression of IR-A and IGF-2 has also been proposed as a potential mechanism that may lead to the resistance to IGF-1R-directed therapies (Hendrickson and Haluska, Curr Opin Investig Drugs. 2009, 10(10):1032-40; Zhang et al., 2007 Cancer Res. 67: 391-397). Numerous preclinical studies have reported that down-regulation of IGF-1R expression or blocking of signaling leads to the inhibition of tumor growth, both in vitro and in vivo (Ryan and Goss, Oncologist. 2008, 13(1):16-24; Sachdev and Yee, Mol Cancer Ther. 2007, 6(1):1-12; Baserga, Expert Opin Ther Targets. 2005, 9(4):753-68). Inhibition of IGF signaling has also been shown to increase the susceptibility of tumor cells to chemotherapeutic agents in vivo (Tao et al., 2007 Nat. Clin. Pract. Oncol. 4:591-602; Chitnis et al., 2008, Clin. Cancer Res. 14: 6364-6370; Ryan and Goss, 2008 Oncologist 13: 16-24; Yuen and Macaulay, 2008 Expert Opin. Ther. Targets 12: 589-603). Dual inhibition of both the IR-A and IGF-1R receptors may enhance therapeutic efficacy against IGF-driven cancers (Sachdev and Yee, Mol Cancer Ther. 2007, 6(1):1-12).

### Sarcoma

Sarcomas are neoplasias from transformed cells of mesenchymal origin, including osteosarcoma, which develops from bone, and soft tissue sarcoma, which develop from soft tissues like fat, muscle, nerves, fibrous tissues, blood vessels, or deep skin tissues. Sarcomas may be named based on the type of tissue that they most closely resemble. For example, osteosarcoma resembles bone, chondrosarcoma resembles cartilage, liposarcoma resembles fat, and leiomyosarcoma resembles smooth muscle. Sarcomas include without limitation Ewing's sarcoma, Osteosarcoma, Rhabdomyosarcoma, Askin's tumor, Sarcoma botryoides, Chondrosarcoma, Malignant Hemangioendothelioma, Malignant Schwannoma, soft tissue sarcoma, Alveolar soft part sarcoma, Angiosarcoma, Cystosarcoma Phyllodes, Dermatofibrosarcoma protuberans, Desmoid Tumor, Desmoplastic small round cell tumor, Epithelioid Sarcoma, Extraskeletal chondrosarcoma, Extraskeletal osteosarcoma, Fibrosarcoma, Hemangiopericytoma, Hemangiosarcoma, Kaposi's sarcoma, Leiomyosarcoma, Liposarcoma, Lymphangiosarcoma, Lymphosarcoma, Malignant peripheral nerve sheath tumor, Neurofibrosarcoma, Synovial sarcoma, and Undifferentiated pleomorphic sarcoma.

An autocrine loop involving IGF-1R and both of its ligands, IGF-1 and IGF-2, has been demonstrated as a key mechanism driving the proliferation and survival of sarcoma cells (Kim et al., 2009 Bull. Cancer 96(7): 52-60). High expression of IGF-1R, IGF-1, or IGF-2 are indicated in most Ewing's sarcomas, osteosarcoma, and rhabdomyosarcoma. Ewing's sarcomas secrete more IGF-1 whereas rhabdomyosarcomas secrete more IGF-2. IGF-1 is highly expressed and stimulates osteosarcoma cell growth. Genetic alterations in the IGF pathway are also prevalent in a number of sarcoma tumors. Loss of imprinting at the IGF-2 locus is commonly detected in embryonal RMS and a genetic alteration that leads to chimeric transcription factors (PAX3-FKHR or PAX7-FKHR) leads to increased expression of IGF-1R in alveolar types of rhabdomyosarcoma. Conversely, in Ewing's sarcoma patients that carry the EWS-FLI1 genetic alteration that upregulates a repressor of IGF-1 signaling, insulin growth factor binding protein 3 (IGFBP3), these patients have improved prognosis. Given the strong disease linkage to the IGF signaling pathway, targeted therapeutic approaches that inhibit the IGF-1R receptor using MAbs have been explored in several types of sarcomas. These IGF-1R-targeted MAbs inhibit IGF-1 and IGF-2 signaling through IGF1R and heterodimeric IGF-1R/IR but do not inhibit IGF-2 signaling through IR-A and thus, may be limited.

### Ewing's Sarcoma

Ewing's sarcoma, peripheral primitive neuroectodermal tumor, and Askin tumor form a group of tumors, collectively termed Ewing's sarcoma family of tumors (ESFT). These tumors are characterized by specific chromosomal translocations that cause the N-terminus of RNA-binding protein EWS to be fused to the C-terminus of one member of the ETS family of transcription factors, most commonly Friend leukemia integration 1 transcription factor (FLI1). Expression of the fusion product has been implicated in oncogenesis.

EFST cell lines express IGF-1R and secrete IGF-1 in an autocrine loop. The prevalence of IGF-1R expression in EFST is very high, with most cell lines and clinical samples positive for expression. In murine fibroblasts, the EWS-FLI1 oncoprotein requires IGF-1R for transformation. Some evidence indicates that relapse-free survival may correlate with the ratio of serum IGFBP-3 to IGF-1. In support of this theory, EWS-FLI1 directly reduces the expression and secretion of IGFBP-3 and exogenous IGFBP-3 inhibits the growth of ESFT cells. Pathways downstream of IGF-1R, including PI3K/Akt and MAPK, are activated and are vital to ESFT cell survival. Inhibitors of both PI3K and MAPK cause growth arrest in ESFT cells.

### Rhabdomyosarcoma

Rhabdomyosarcoma is the most common soft tissue sarcoma of childhood, arising from developing cells that form striated muscle. IGF-2 is involved in normal muscle growth, and analysis of tumor biopsy specimens from patients with rhabdomyosarcoma demonstrated high levels of IGF-2 mRNA expression. Without being bound to a particular theory, upregulation of IGF-2 potentially plays a role in the unregulated growth of these tumors. Additionally, it has been observed that binding of IGF-1R and IGF-2 secreted from rhabdomyosarcoma cell lines, resulted in autocrine growth proliferation and increased cell motility.

Epigenetic changes leading to loss of imprinting (LOI) of the IGF-2 locus, resulting in over-expression of IGF-2, have been identified. In addition, the PAX3-FKHR translocation that characterizes certain rhabdomyosarcomas transactivates the IGF-1R promoter, thus providing further evidence that the IGF pathway plays an important role in the progression of rhabdomyosarcoma. All rhabdomyosarcoma cell lines show some level of IGF-1R expression, although they may differ by as much as 30-fold based on quantitative protein analysis.

### Osteosarcoma

The peak incidence of osteosarcoma occurs during adolescence, corresponding to both the growth spurt and peak concentrations of circulating GH and IGF-1. High levels of IGF-1 appear to play an important role in the pathogenesis of osteosarcoma. Preclinical data indicate a role for IGF-1 in osteosarcoma. Osteosarcoma cells express functional IGF-1R on the cell surface, and the majority of osteosarcoma patient samples express IGF ligands and 45% express IGF-1R. Exogenous IGF-1 stimulates proliferation of osteosarcoma cells, and IGF-1-dependent growth can be inhibited using monoclonal antibodies or antisense oligonucleotides against IGF-1R. Furthermore, treatment of mice using a humanized anti-IGF-1R antibody resulted in tumor regression in two osteosarcoma xenograft models.

Mammalian Target of Rapamycin (mTOR)

The mammalian target of rapamycin (mTOR) is a serine/threonine protein kinase that plays an important role in regulating cell growth, proliferation, and survival. mTOR integrates the input from upstream pathways, including insulin, growth factors (such as IGF-1 and IGF-2), and amino acids. mTOR also senses cellular nutrient, oxygen, and energy levels. The mTOR pathway is dysregulated in human diseases, such as diabetes, obesity, depression, and certain cancers. mTOR was identified as being sensitive to the antifungal agent rapamycin. Rapamycin is a bacterial product that can inhibit mTOR by associating with its intracellular receptor FKBP12. The FKBP12-rapamycin complex binds directly to the FKBP12-Rapamycin Binding (FRB) domain of mTOR, inhibiting its activity.

Activation of mTOR leads to phosphorylation of downstream Ribosomal protein S6 kinase beta-1 (S6K) and Eukaryotic translation initiation factor 4E-binding protein 1 (4E-BP1). mTOR signaling has been an attractive therapeutic target for cancer therapy. mTOR inhibitors Temsirolimus and Everolimus have been approved for treating metastatic renal cell carcinoma and pancreatic neuroendocrine tumors respectively. Ridaforolimus is currently in phase III trial in sarcoma patients. However, rapamycin and its derivatives induce Akt activation by releasing the negative feedback between S6K and IRS/PI3K, and subsequently reactivating IGF-1R signaling. This contributes to the possible mechanism of resistance to mTOR inhibitors, and suggests a potential benefit of combining rapamycin with agents targeting IGF pathway. Combination of several IGF-1R targeting agents with different rapamycin analogs are in early phase clinical trials. First generation mTOR inhibitors include without limitation rapamycin, temsirolimus (CCI-779), everolimus (RAD001), ridaforolimus (AP-23573). Second generation mTOR inhibitors are designed to compete with ATP in the catalytic site of mTOR. Such ATP-competitive mTOR kinase inhibitors include without limitation AZD2014, INK128, AZD8055, NVP-BEZ235, BGT226, SF1126, PKI-587. Structures of mTOR inhibitors AZD2014 and rapamycin are provided below.

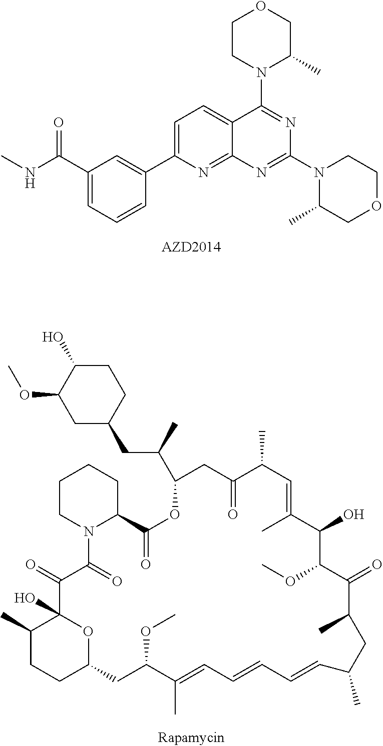 [SMILES: CNC(=O)c1cccc(-c2ccc3c(N4CCOC[C@@H]4C)nc(N4CCOC[C@@H]4C)nc3n2)c1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

### Antibodies

Antibodies that selectively bind IGF-1/-2 and inhibit the binding or activation of receptors to of IGF-1/-2 are useful in the methods of the invention. In certain embodiments, the antibodies to IGF-1/-2 do not bind insulin or inhibit the biological activity of insulin.

In an embodiment, the antibody is a recombinant, monoclonal antibody. The recombinant monoclonal antibody is prepared from a host cell, including, but not limited to, a bacterial cell, a yeast cell, an insect cell, or a mammalian cell. In a preferred embodiment, the host cell is a mammalian cell. In another embodiment, the recombinant monoclonal antibody is a human antibody. In yet another embodiment, the monoclonal antibody is an IgA, IgE, IgD, IgE, or IgG antibody. In a preferred embodiment, the monoclonal antibody is an IgG antibody, including, but not limited to an IgG1 or IgG2 antibody.

In another embodiment, the antibody comprises at least one N-linked glycosylation site on the Fc region of the antibody and at least one N-linked glycosylation site on the Fab region of the antibody. In another embodiment, the antibody has only one N-linked glycosylation site on the Fc region of the antibody and only one N-linked glycosylation site on the Fab region of the antibody (i.e., at total of 3 N-linked glycosylation sites).

Antibodies can be made by any of the methods known in the art.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies.

One method for generating fully human antibodies is through the use of XenoMouse® strains of mice that have been engineered to contain up to but less than 1000 kb-sized germline configured fragments of the human heavy chain locus and kappa light chain locus. See Mendez et al. Nature Genetics 15: 146-156 (1997) and Green and Jakobovits J. Exp. Med. 188:483-495 (1998). The XenoMouse® strains are available from Abgenix, Inc. (Fremont, Calif.).

The production of the XenoMouse® strains of mice is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, filed Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, Ser. No. 08/759,620, filed Dec. 3, 1996, U.S. Publication 2003/0093820, filed Nov. 30, 2001 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and usually a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625, 126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B 1, International Patent Application Nos. WO 92/03918, WO 92/22645, WO 92/22647, WO92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference. Additionally, KM™-mice, which are the result of cross-breeding of Kirin's Tc mice with Medarex's minilocus (Humab) mice have been generated. These mice possess the human IgH transchromosome of the Kirin mice and the kappa chain trans gene of the Genpharm mice (Ishida et al., Cloning Stem Cells, (2002) 4:91-102).

Human antibodies can also be derived by in vitro methods. Suitable examples include but are not limited to phage display (CAT, Morphosys, Dyax, Biosite/Medarex, Xoma, Symphogen, Alexion (formerly Proliferon), Affimed) ribosome display (CAT), yeast display, and the like.

Antibodies, as described herein, were prepared through the utilization of the XenoMouse® technology, as described below. Such mice, then, are capable of producing human immunoglobulin molecules and antibodies and are deficient in the production of murine immunoglobulin molecules and antibodies. Technologies utilized for achieving the same are disclosed in the patents, applications, and references disclosed in the background section herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15: 146-156 (1997), the disclosure of which is hereby incorporated by reference.

Through the use of such technology, fully human monoclonal antibodies to a variety of antigens have been produced. Essentially, XenoMouse® lines of mice are immunized with an antigen of interest (e.g. IGF-17II), lymphatic cells (such as B-cells) are recovered from the hyper-immunized mice, and the recovered lymphocytes are fused with a myeloid-type cell line to prepare immortal hybridoma cell lines. These hybridoma cell lines are screened and selected to identify hybridoma cell lines that produced antibodies specific to the antigen of interest. Provided herein are methods for the production of multiple hybridoma cell lines that produce antibodies specific to IGF-1/-2. Further, provided herein are characterization of the antibodies produced by such cell lines, including nucleotide and amino acid sequence analyses of the heavy and light chains of such antibodies.

Alternatively, instead of being fused to myeloma cells to generate hybridomas, B cells can be directly assayed. For example, CD19⁺ B cells can be isolated from hyperimmune XenoMouse® mice and allowed to proliferate and differentiate into antibody-secreting plasma cells. Antibodies from the cell supematants are then screened by ELISA for reactivity against the IGF-1/-2 immunogen. The supematants might also be screened for immunoreactivity against fragments of IGF-1/-2 to further map the different antibodies for binding to domains of functional interest on IGF-17II. The antibodies may also be screened against other related human chemokines and against the rat, the mouse, and non-human primate, such as cynomolgus monkey, orthologues of IGF-1/-2, the last to determine species cross-reactivity. B cells from wells containing antibodies of interest may be immortalized by various methods including fusion to make hybridomas either from individual or from pooled wells, or by infection with EBV or transfection by known immortalizing genes and then plating in suitable medium. Alternatively, single plasma cells secreting antibodies with the desired specificities are then isolated using an IGF-1/-2-specific hemolytic plaque assay (Babcook et al., Proc. Natl. Acad. Sci. USA 93:7843-48 (1996)). Cells targeted for lysis are preferably sheep red blood cells (SRBCs) coated with the IGF-1/-2 antigen.

In the presence of a B-cell culture containing plasma cells secreting the immunoglobulin of interest and complement, the formation of a plaque indicates specific IGF-1/-2-mediated lysis of the sheep red blood cells surrounding the plasma cell of interest. The single antigen-specific plasma cell in the center of the plaque can be isolated and the genetic information that encodes the specificity of the antibody is isolated from the single plasma cell. Using reverse-transcription followed by PCR (RT-PCR), the DNA encoding the heavy and light chain variable regions of the antibody can be cloned. Such cloned DNA can then be further inserted into a suitable expression vector, preferably a vector cassette such as a pcDNA, more preferably such a pcDNA vector containing the constant domains of immunglobulin heavy and light chain. The generated vector can then be transfected into host cells, e.g., HEK293 cells, CHO cells, and cultured in conventional nutrient media modified as appropriate for inducing transcription, selecting transformants, or amplifying the genes encoding the desired sequences.

In general, antibodies produced by the fused hybridomas were human IgG2 heavy chains with fully human kappa or lambda light chains. Antibodies described herein possess human IgG4 heavy chains as well as IgG2 heavy chains. Antibodies can also be of other human isotypes, including IgG1. The antibodies possessed high affinities, typically possessing a K_{d} of from about 10⁶ through about 10¹² M or below, when measured by solid phase and solution phase techniques. Antibodies possessing a KD of at least 10¹¹ M are desired to inhibit the activity of IGF-1/-2.

As will be appreciated, anti-IGF-1/-2 antibodies can be expressed in cell lines other than hybridoma cell lines. Sequences encoding particular antibodies can be used to transform a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Cell lines of particular preference are selected through determining which cell lines have high expression levels and produce antibodies with constitutive IGF-1/-2 binding properties.

In other embodiments, the invention provides “unconventional antibodies.” Unconventional antibodies include, but are not limited to, nanobodies, linear antibodies (Zapata et al., Protein Eng. 8(10): 1057-1062, 1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of .about.60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In one embodiment, the antibody binds to insulin-like growth factor 2 (IGF-2) with cross reactivity to insulin-like growth factor 1 (IGF-1), such as those antibodies disclosed in U.S. Pat. No. 7,939,637, which is hereby incorporated by reference in its entirety. In certain embodiments, the antibody binds to IGF-2 with cross reactivity to IGF-1 and is a monoclonal, human antibody selected from the group consisting of mAb 7.251.3 (ATCC Accession Number PTA-7422), mAb 7.34.1 (ATCC Accession Number PTA-7423), and mAb 7.159.2/MEDI-573 (ATCC Accession Number PTA-7424).

In particular embodiments of the invention, the antibody in the pharmaceutical composition comprises a heavy chain complementarity determining region 1 (CDR1) comprising the amino acid sequence set forth in SEQ ID NO: 1 (Ser Tyr Asp Ile Asn); a heavy chain complementarity determining region 2 (CDR2) comprising the amino acid sequence set forth in SEQ ID NO: 2 (Trp Met Asn Pro Asn Ser Gly Asn Thr Gly Tyr Ala Gln Lys Phe Gln Gly); a heavy chain complementarity determining region 3 (CDR3) comprising the amino acid sequence set forth in SEQ ID NO: 3 (Asp Pro Tyr Tyr Tyr Tyr Tyr Gly Met Asp Val); a light chain complementarity determining region 1 (CDR1) comprising the amino acid sequence set forth in SEQ ID NO: 4 (Ser Gly Ser Ser Ser Asn Ile Glu Asn Asn His Val Ser); a light chain complementarity determining region 2 (CDR2) comprising the amino acid sequence set forth in SEQ ID NO: 5 (Asp Asn Asn Lys Arg Pro Ser); and a light chain complementarity determining region 3 (CDR3) comprising the amino acid sequence set forth in SEQ ID NO: 6 (Glu Thr Trp Asp Thr Ser Leu Ser Ala Gly Arg Val).

In some embodiments, the antibody in the pharmaceutical composition of the invention comprises one or more variable regions comprising an amino acid sequence selected from the amino acid sequences set forth in SEQ ID NO: 7 and SEQ ID NO: 8. In particular embodiments, the antibody in the pharmaceutical composition of the invention has the amino acid sequence of the antibody produced by hybridoma cell line 7.159.2 (ATCC Accession Number PTA-7424).

MEDI-573 is a fully human immunoglobulin G2 lambda (IgG2) antibody generated with Xenomouse® technology and manufactured in Chinese Hamster Ovary (CHO) cells. MEDI-573 selectively binds to human insulin-like growth factors hIGF-1 and hIGF-2 and inhibits insulin-like growth factor IGF-1 and IGF-2 mediated signal transduction in tumor cells, thereby inhibiting tumor growth. The antibody was isolated from mice immunized alternately with soluble recombinant human hIGF-1 and hIGF-2 coupled to keyhole limpet hemocyanin (KLH), as described in U.S. Pat. No. 7,939,637, which is herein incorporated by reference in its entirety. MEDI-573 is composed of 2 light chains and 2 heavy chains, with an overall molecular weight of approximately 151 kilodaltons.

MEDI 573 selectively binds to human insulin-like growth factor (hIGF)-I and hIGF-2 and IGF-1- and IGF-2 mediated signal transduction and proliferation in human tumor cells. MEDI-573 targets the IGF-1 and IGF-2 ligands and thereby inhibits IGF-mediated signal transduction. Nonclinical studies in human cancer cells suggest that MEDI 573 has the potential to achieve broad antitumor efficacy owing to its ability to inhibit both IGF-1R and IR-A pathways. Furthermore, MEDI-573 has potential to achieve this without perturbing glucose homeostasis, which has been an adverse effect observed with investigational agents that target IGF 1R. The results of in vitro studies have shown that MEDI-573 inhibited both IGF-1 and IGF-2-stimulated phosphorylation of IGF 1R and that of downstream signaling proteins including Akt and MAPK in a number of engineered mouse embryonic fibroblast NIH-3T3 cell lines transfected to express human IGF-1R and either human IGF-1/-2. Furthermore, MEDI-573 inhibited autocrine phosphorylation of these signaling molecules. Functionally, MEDI-573 effectively inhibited the growth of a number of engineered NIH3T3 and human tumor cell lines in vitro. In vivo, treatment of tumor-bearing mice with MEDI-573 significantly inhibited the growth of implanted clone 32 (C32) and clone P12 (P12) tumors, which overexpress hIGF II and human insulin-like growth factor 1 receptor (hIGF-1R), and hIGF-1 and hIGF-1R, respectively.

### Therapy

Therapy may be provided wherever cancer therapy is performed: at home, the doctor's office, a clinic, a hospital's outpatient department, or a hospital. In one embodiment, the invention provides for the use of an anti-IGF-1/-2 antibody (e.g., MEDI-573) in combination with an mTOR inhibitor as a therapy.

Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place. Cancer growth is uncontrolled and progressive, and occurs under conditions that would not elicit, or would cause cessation of, multiplication of normal cells.

As described above, if desired, treatment with a composition of the invention may be combined with therapies for the treatment of proliferative disease (e.g., radiotherapy, surgery, or chemotherapy).

### Formulation of Pharmaceutical Compositions

The administration of a combination of the invention (e.g., an antibody that binds IGF-1/-2 with an mTOR inhibitor) for the treatment of sarcoma may be by any suitable means that results in a concentration of the therapeutic that, combined with other components, is effective in preventing, ameliorating, or reducing sarcoma. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in a sarcoma (v) formulations that allow for convenient dosing, such that doses are administered, for example, once every one or two weeks; and (vi) formulations that target proliferating neoplastic cells by using carriers or chemical derivatives to deliver the therapeutic agent to a sarcoma cell. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

A composition of the invention, may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic.

Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols. For any of the methods of application described above, a composition of the invention is desirably administered intravenously or is applied to the site of the needed apoptosis event (e.g., by injection).

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for delivering agents include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. The preferred dosage of a composition of the invention is likely to depend on such variables as the type and extent of the disorder, the overall health status of the particular patient, the formulation of the compound excipients, and its route of administration.

Human dosage amounts for any therapy described herein can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher doses may be used, such doses may be in the range of about 5 mg compound/Kg body to about 20 mg compound/Kg body. In other embodiments the doses may be about 8, 10, 12, 14, 16 or 18 mg/Kg body weight. Of course, a dosage amount may be adjusted upward or downward, as is routinely done in such treatment protocols, depending on the results of the initial clinical trials and the needs of a particular patient.

In certain embodiments, dosages include at least two doses of an antibody which binds IGF-1 and/or IGF-2. The doses are separated by about a week, or by about three weeks, and each dose comprises an amount of antibody greater than about 0.5 mg kg of patient body mass and less than about 50 mg per kg of patient body mass. Dosing with regard to MEDI-573, is described for example in WO2012068148, which is herein incorporated in its entirety.

### Kits

The invention provides kits for the treatment or prevention of sarcoma. In an embodiment, the kit includes a therapeutic or prophylactic composition containing an effective amount of an antibody and one or more mTOR inhibitors. The antibody may specifically bind IGF-1 and/or IGF-2 and may inhibit their activity. In an embodiment, the antibody may be MEDI-573. In an embodiment, the mTOR inhibitor may be one or more of AZD2014, INK128, AZD8055, NVP-BEZ235, BGT226, SF1126, PKI-587, rapamycin, temsirolimus, everolimus, ridaforolimus, and combinations thereof. In a particular embodiment, the mTOR inhibitor is rapamycin. In a particular embodiment, the mTOR inhibitor is AZD2014. In a particular embodiment, the kit includes a therapeutic or prophylactic composition containing an effective amount of MEDI-573 and rapamycin in unit dosage form. In a particular embodiment, the kit includes a therapeutic or prophylactic composition containing an effective amount of MEDI-573 and aAZD2014 in unit dosage form.

In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The antibody of the invention may be provided together with instructions for administering the antibody and mTOR inhibitor to a subject having or at risk of developing sarcoma. The instructions may generally include information about the use of the composition for the treatment or prevention of sarcoma. In other embodiments, the instructions include at least one of the following: description of the therapeutic agent; dosage schedule and administration for treatment or prevention of sarcoma or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

### Example 1

### IGF-1, IGF-2, and IGF-1R Levels and IR-A:IR-B Ratio in Sarcoma Xenografts and Cells

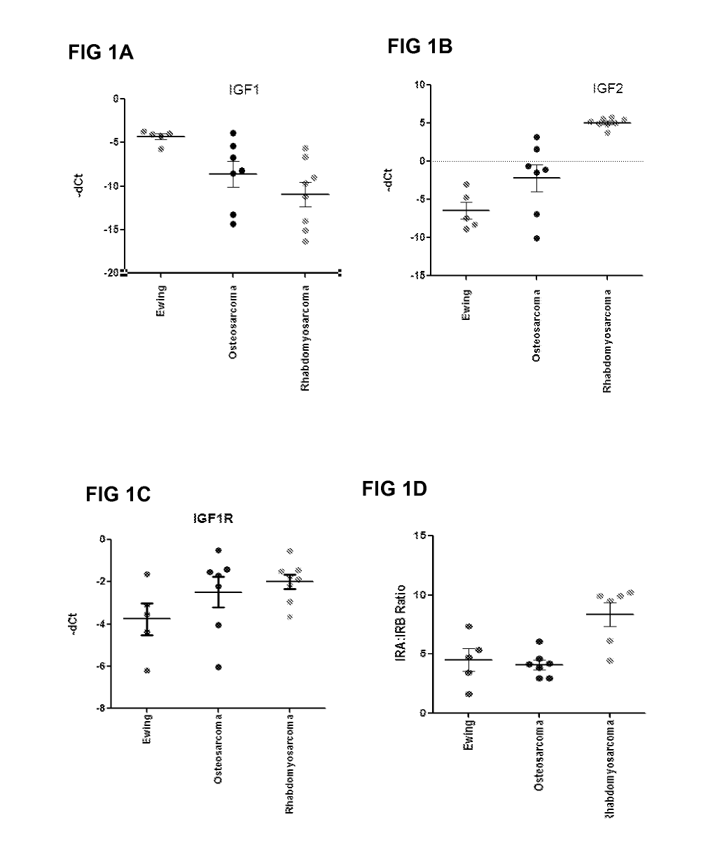

mRNA levels of Insulin-like growth factor 1 (IGF-1), Insulin-like growth factor 2 (IGF-2), and Insulin-like growth factor 1 receptor (IGF-1R) in 23 xenografts from pediatric sarcomas (age: 6 months to 25 years) were determined by qRT-PCR. The results of these analyses are shown in FIG. 1A to FIG. 1D. As seen in FIG. 1A, the mRNA levels of IGF-1 were found to be significantly higher in Ewing's sarcomas than in osteosarcomas (p=0.029) and rhabdomyosarcomas (p=0.0024). In contrast, as seen in FIG. 1B, the mRNA levels of IGF-2 were found to be significantly higher in rhabdomyosarcomas than in Ewing's sarcomas (p=0.0005) and osteosarcomas (p=0.0066). All 3 subtypes of sarcoma expressed high mRNA levels of IGF-1R, shown in FIG. 1C. The majority of sarcoma xenograft samples assayed had a high cycle threshold (ΔCt) differential in the ratio of insulin receptor A isoform to insulin receptor B isoform (IR-A:IR-B) (ΔCt<−4), with rhabdomyosarcomas being the highest (FIG. 1D).

Also using qRT-PCR, the mRNA levels of IGF ligands and receptors were measured in a number of sarcoma cell lines including Ewing's sarcoma, rhabdosarcoma, and osteosarcoma.

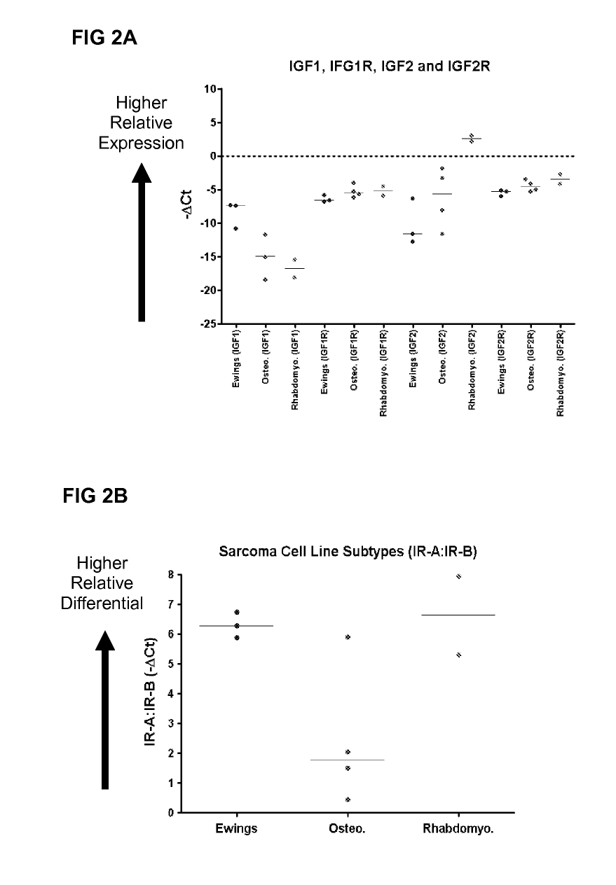

The results are shown in Table 1, below. Consistent with the results in xenograft samples, Ewing's sarcoma cells had the highest IGF-1 levels, while rhabdomyosarcoma cells expressed the highest IGF-2 levels. A graph depicting the calculated ΔCt, for IGF-1, IGF-1R, IGF-2, and IGF2R is depicted in FIG. 2A. The calculated ΔCt for the IR-A:IR-B ratio is depicted in FIG. 2B.

TABLE 1
mRNA Levels of IGF Ligands and Receptors

| Cell line/-dCT | IGF1 | IGF1R | IGF2 | IGF2R | IRA:IRB | INSR | IRA | IRB | Subtype |
|---|---|---|---|---|---|---|---|---|---|
| A673 | −7.28 | −6.74 | −11.56 | −5.29 | 5.89 | −9.32 | −7.19 | −13.09 | Ewings |
| RD-ES | −10.81 | −5.81 | −6.28 | −5.12 | 6.28 | −8.86 | −6.51 | −12.79 | Ewings |
| SKES1 | −7.38 | −6.59 | −12.69 | −6.01 | 6.73 | −7.69 | −5.66 | −12.39 | Ewings |
| KHO S | N/A | −5.61 | −1.85 | −5.23 | 2.05 | −10.55 | −8.97 | −11.02 | Osteo |
| MG−63 | −18.42 | −5.26 | −11.59 | −5.05 | 0.45 | −7.47 | −6.47 | −6.92 | Osteo |
| SA0S2 | −14.94 | −3.97 | −8.04 | −3.39 | 5.91 | −8.10 | −6.12 | −12.03 | Osteo |
| SJSA−1 | −11.68 | −6.12 | −3.24 | −4.17 | 1.50 | −10.35 | −8.63 | −10.12 | Osteo |
| RD | −18.03 | −5.88 | 2.15 | −4.16 | 5.31 | −9.24 | −7.19 | −12.51 | Rhabdo |
| SJCRH30 | −15.42 | −4.51 | 3.01 | −2.74 | 7.95 | −7.09 | −4.89 | −12.85 | Rhabdo |

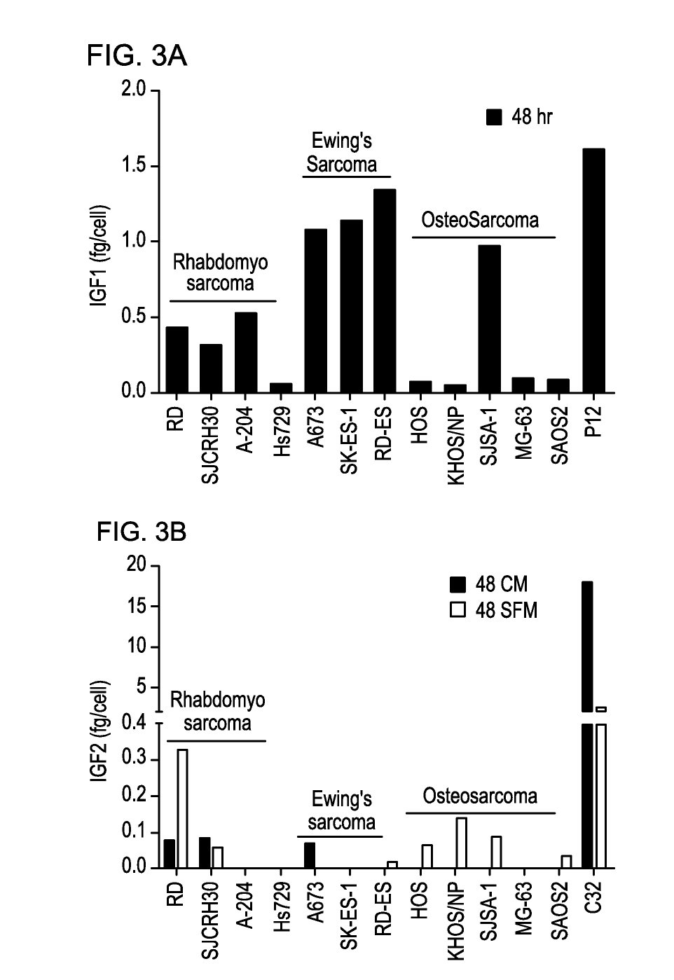

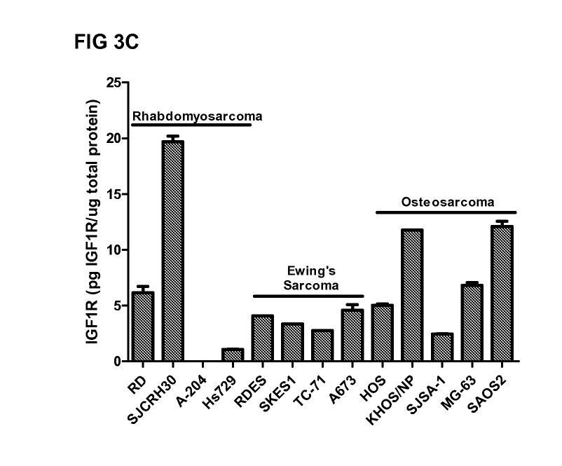

The protein levels of IGF-1, IGF-2, and IGF-1R were determined by ELISA in the same sarcoma cell lines. These results are depicted in FIG. 3A to FIG. 3C. The results showed that most sarcoma cell lines expressed IGF-1R and IGF-1 proteins (FIG. 3A and FIG. 3C). Only osteosarcoma cell lines and a few rhabdosarcoma cell lines secreted IGF-2 (FIG. 3B). None of the Ewing's sarcoma cell lines expressed detectable amounts of IGF-2.

### Example 2

### MEDI-573 Inhibited Sarcoma Cell Growth and Proliferation Driven by Autocrine or Paracrine IGF Ligands

To determine the effect of treatment with MEDI-573 antibody on the growth of tumor cells, three rhabdomyosarcoma cell lines (RD, SJCRH30, and Hs729); three Ewing's sarcoma cell lines (RD-ES, TC-71, and SK-ES-1), and four osteosarcoma cell lines (SJSA-1, KHOS, MG-63, and SAOS2) were treated with MEDI-573, an anti-IGF antibody, in the absence of exogenous IGF-1 or IGF-2.

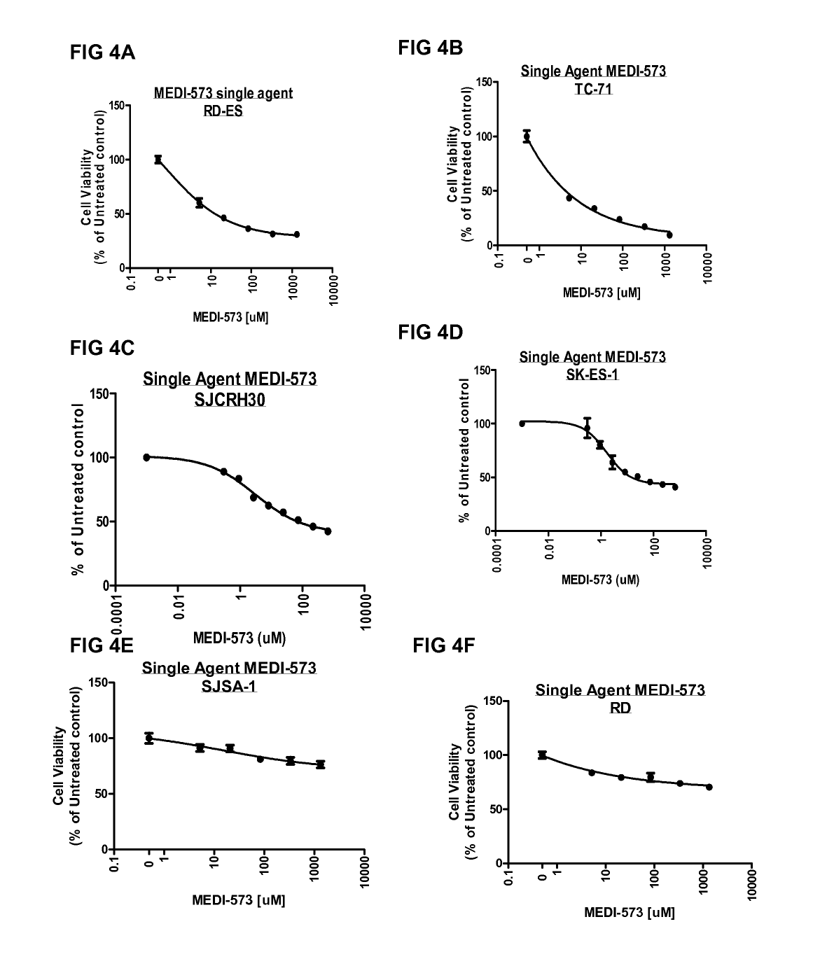

The growth of all three Ewing's sarcoma cell lines (RD-ES, TC-71, and SK-ES-1) and one rhabdomyosarcoma cell line (SJCRH30) was inhibited by the MEDI-573 antibody in the absence of exogenous IGF-1 or IGF-2. Without being bound to a particular theory, this result indicates that these lines secrete endogenous IGF-1 or IGF-2 to drive their growth (autocrine driven). There was moderate growth inhibition (˜30% at highest dose tested) in the RD and SJSA-1 cells. The results are depicted in Table 2, below, and in FIG. 4A to FIG. 4F, where FIG. 4A depicts a graph of the cell viability of the RD-ES cells treated with MEDI-573; FIG. 4B depicts a graph of the cell viability of the TC-71 cells treated with MEDI-573; FIG. 4C depicts a graph of the cell viability of the SJCRH30 cells treated with MEDI-573; FIG. 4D depicts a graph of the cell viability of the SK-ES-1 cells treated with MEDI-573; FIG. 4E depicts a graph of the cell viability of the SJSA-1 cells treated with MEDI-573; and FIG. 4F depicts a graph of the cell viability of the RD cells treated with MEDI-573.

TABLE 2
Effect of the addition of MEDI-573 to different cell lines

| Sarcoma Subtype | Cell Line | IC50 (μM) |
|---|---|---|
| Rhabdomyosarcoma | RD | 30% inhibition |
| | SJCRH30 | 3.2 μM |
| | Hs729 | Inactive |
| Ewing's Sarcoma | RD-ES | 6.9 μM |
| | TC-71 | 2.7 μM |
| | SK-ES-1 | 3.2 μM |
| Osteosarcoma | SJSA-1 | 30% inhibition |
| | KHOS | Inactive |
| | MG-63 | Inactive |
| | SAOS2 | Inactive |

To determine if addition of IGF had an effect on the anti-proliferative activity of MEDI-573, the assay was repeated in a number of sarcoma cell lines that were stimulated with exogenously added IGFs. The results of these assays are shown in Table 3 below.

TABLE 3
Effect of the addition of MEDI-573 to cells stimulated with IGFs

| | | IGF-1 | IGF-2 |
|---|---|---|---|
| Sarcoma Subtype | Cell Line | IC₅₀ (μM) | IC₅₀ (μM) |
| Rhabdomyosarcoma | RD | No Induction | No Induction |
| Ewing's Sarcoma | SK-ES-1 | 20 μM | 20 μM |
| | RD-ES | 40 μM | 2 μM |
| | SJSA-1 | No Induction | No Induction |
| | KHOS | No Induction | No Induction |
| | MG-63 | 223 μM | 5.8 μM |
| | SAOS2 | 177 μM | 5.3 μM |

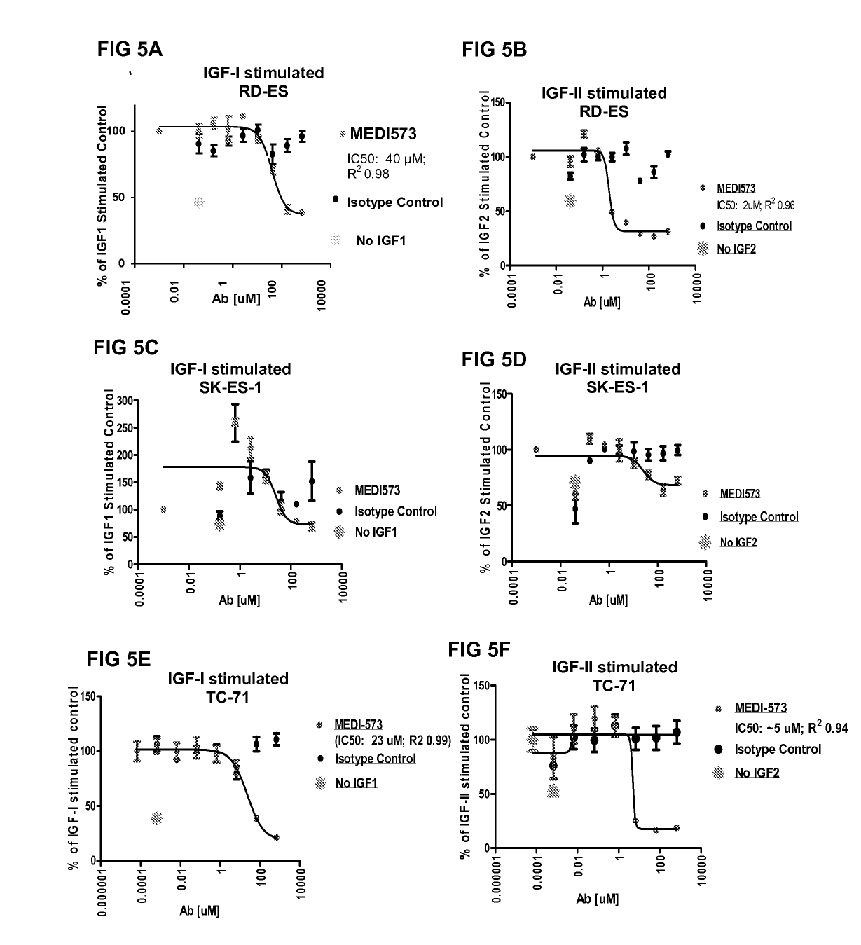

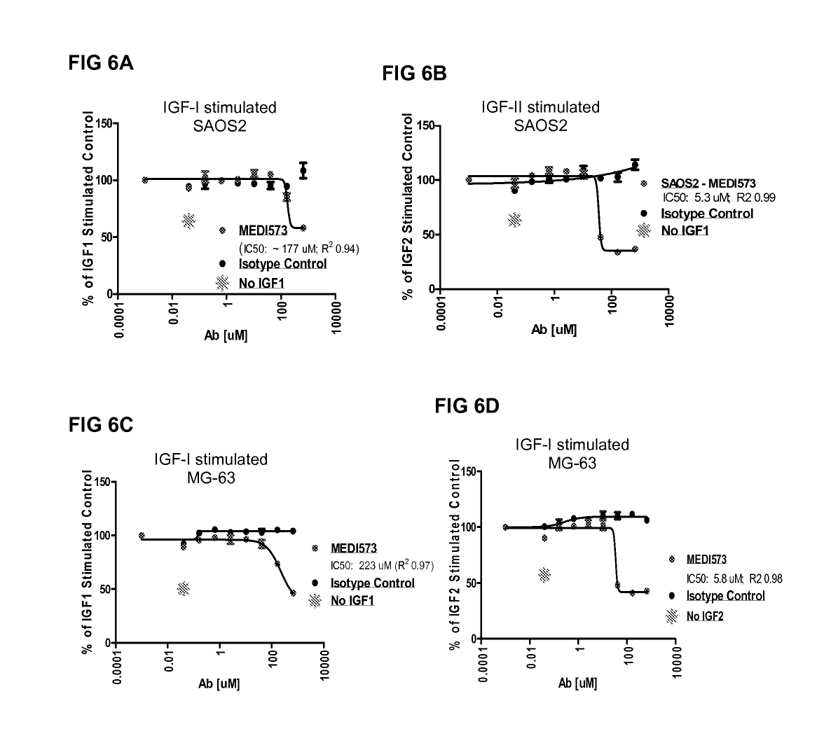

The data from Table 3 is shown in FIG. 5A to FIG. 5F and FIG. 6A to FIG. 6D. The table and figures show that addition of IGF-1 induced cell proliferation in Ewing's sarcoma cell lines RD-ES (FIG. 5A), TC-71 (FIG. 5E), and SK-ES-1 (FIG. 5C) by about 2 fold. Similarly, addition of IGF-2 induced cell proliferation in Ewing's sarcoma cell lines RD-ES (FIG. 5B), TC-71 (FIG. 5F), and SK-ES-1 (FIG. 5D) by about 2 fold. Addition of IGF-1 induced cell proliferation in osteosarcoma cell lines MG-63 (FIG. 6C), and SAOS-2 (FIG. 6A). MEDI-573 potently inhibited IGF-1- and IGF-2-stimulated cell growth. In a relative comparison, MEDI-573 exhibited greater effect against IGF-2-stimulated proliferation (IC₅₀ ranged from 2 to 20 μM) than the IGF-1-stimulated proliferation (IC₅₀ ranged from 20 to 223 μM). Some cell lines, such as KHOS and RD cells, did not respond to IGF-1 or IGF-2 stimulation. MEDI-573 failed to have any significant effect in modulating the growth of KHOS and RD cells with or without IGF stimulation. Without being bound to a particular theory, this indicated that IGF signaling does not drive growth or survival in these unresponsive lines.

To evaluate the basis for the cytotoxic effect of MEDI-573 on RD-ES, TC-71, SJSA-1, and KHOS cells, cells were treated with increasing concentrations of MEDI-573 for 48 hours and analyzed by measuring the activation of caspase-3 and caspase-7. In RD-ES, TC-71, and SJSA-1 cells, treatment with MEDI-573 increased caspase-3/-7 activities in a dose-dependent manner, compared to isotype control. No activation of caspase-3/-7 was detected in KHOS cells. (Data not shown.)

### Example 3

### MEDI-573 Inhibited Tumor Growth in Sarcoma Xenograft Models

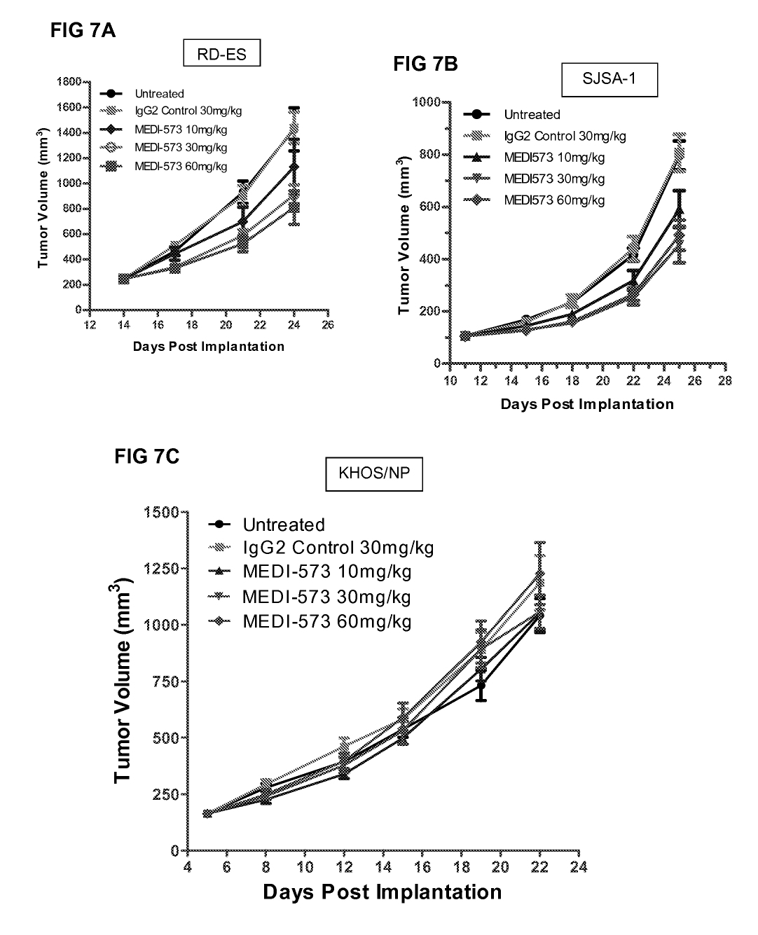

Treatment twice weekly with MEDI-573 of mice bearing RD-ES (Ewing's sarcoma) xenografts resulted in tumor growth inhibition of 25% at 10 mg/kg, 44% at 30 mg/kg, and 52% at 60 mg/kg (FIG. 7A). Similar effects were seen when mice bearing TC-71 xenografts (another Ewing's sarcoma model) were treated in the same manner. Comparable results were obtained when treating with MEDI-573 mice bearing SJSA-1 (an osteosarcoma model) xenografts (FIG. 7B). Although proliferation of SK-ES-1 and SJCRH30 cells was inhibited by MEDI-573 in vitro in the absence of exogenous IGFs, the in vivo growth of these two models was not effected by MEDI-573 treatment. Consistent with the in vitro finding, KHOS cells did not respond to MEDI-573 in vivo either (FIG. 7C). MEDI-573 treatment was well-tolerated in mice as no loss of body weight was observed.

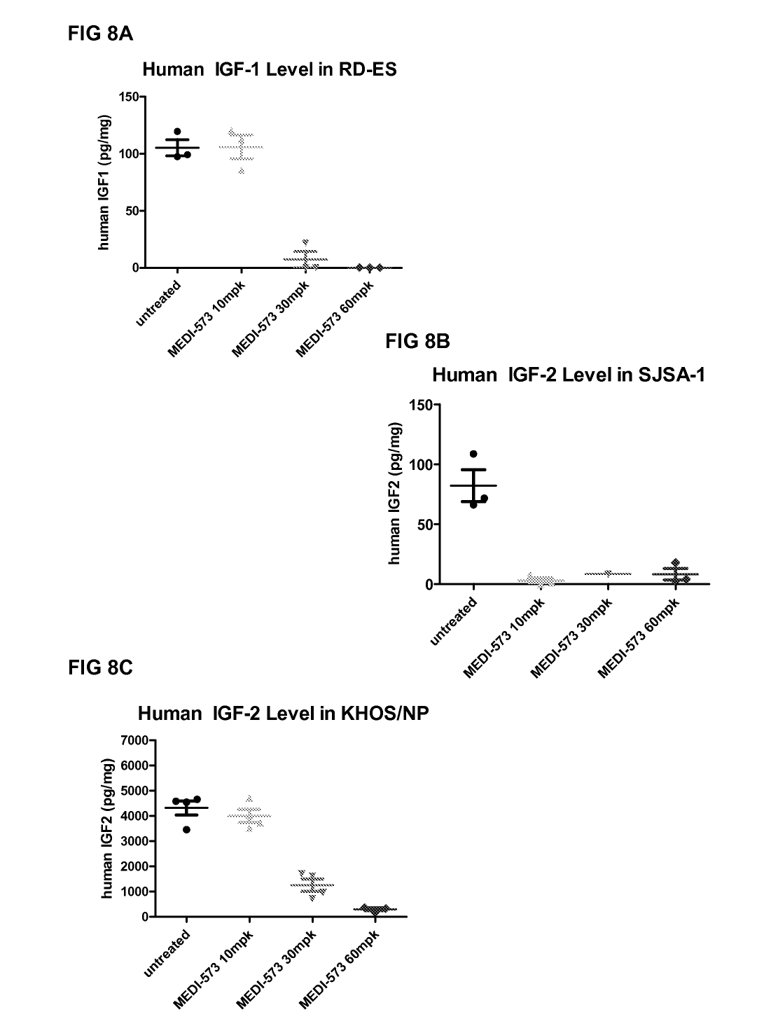

Free IGF ligands were measured in xenograft tumors in untreated mice and in mice treated with different amounts of MEDI-573. In RD-ES tumors, there was a MEDI-573 dose-dependent suppression of IGF-1 (FIG. 8A) and the levels of IGF-2 were too low to be detected. In contrast, SJSA-1 tumors showed detectable levels of IGF-2 (FIG. 8B), but not IGF-1 (data not shown). The free IGF-2 in SJSA-1 tumors was almost completely neutralized by MEDI-573 even at the lowest dose of 10 mg/kg. This may reflect the higher binding affinity of MEDI-573 for IGF-2 (K_{d}=2 pmol/L) compared to IGF-1 (K_{d}=294 pmol/L). Despite KHOS cells being unresponsive to IGF-1 and/or IGF-2 stimulation, IGF-2 levels were examined in a KHOS/NP model. Dose-dependent inhibition of human IGF-2 levels in KHOS/NP model was observed, but some levels of free IGF-2 were detectable even at the highest 60 mg/kg dose, which was comparable to the baseline IGF-2 levels in SJSA-1 tumors (FIG. 8C).

### Example 4

### MEDI-573 Inhibited IGF Signaling in Sarcoma Cells

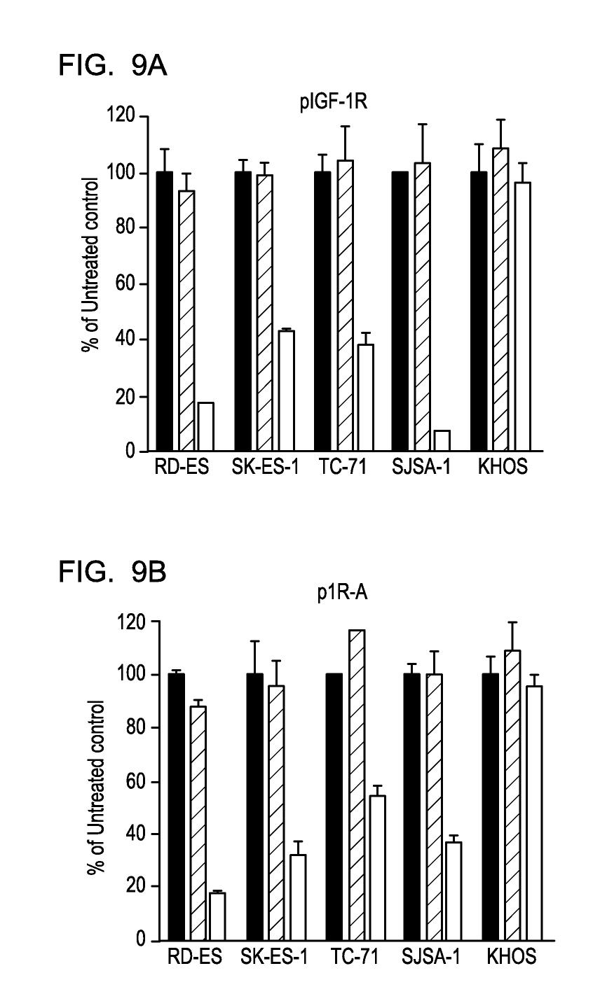

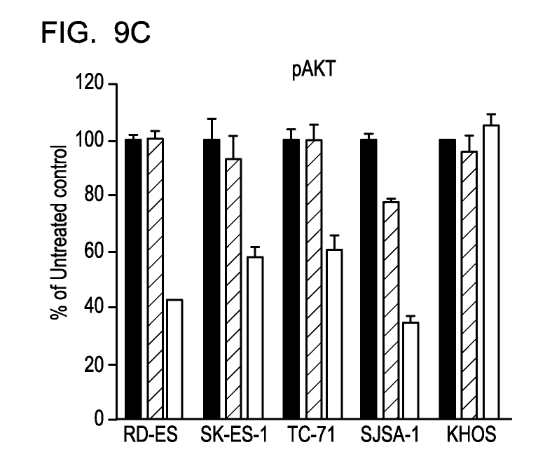

MEDI-573 inhibited autophosphorylation of IGF-1R, IR-A, and Protein Kinase B (Akt) in RD-ES, TC-71, SK-ES-1, and SJSA-1 cells, but not in KHOS cells (FIG. 9A-FIG. 9C).

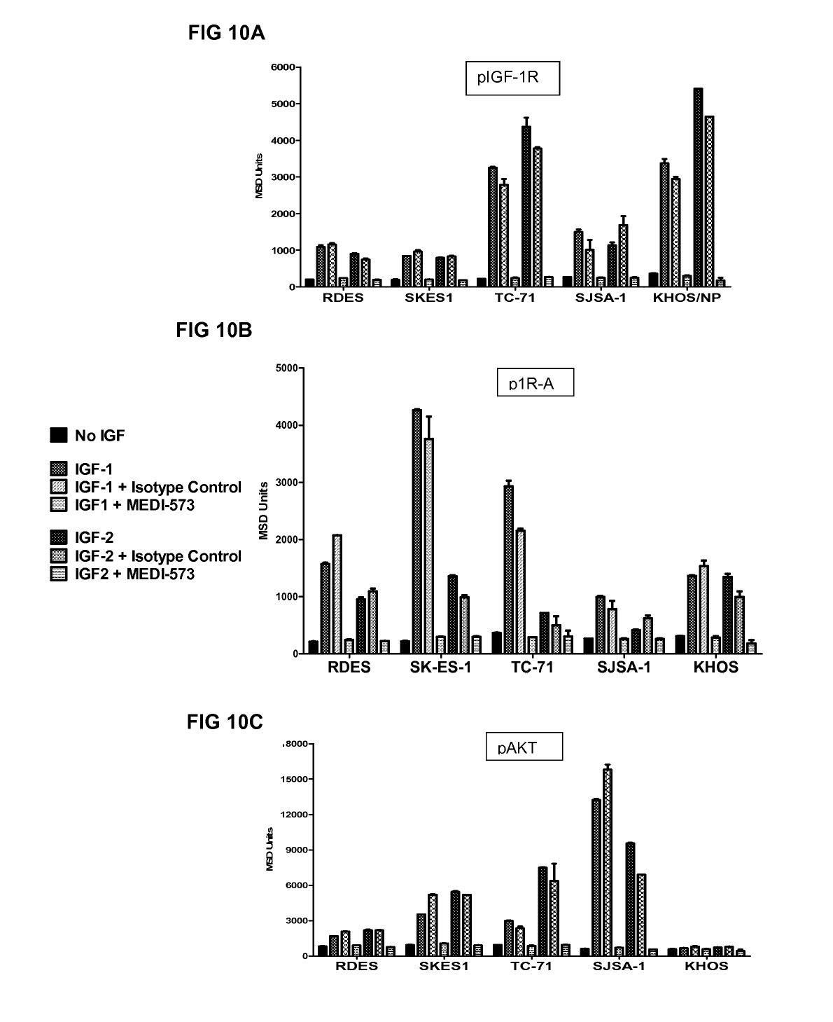

When exogenous IGF-1 or IGF-2 was added to cells, there was an induction of phosphorylation of IGF-1R and IR-A in all cells examined. As seen on FIG. 10 to FIG. 10C, pretreatment with MEDI-573 inhibited IGF-1/-2-induced activation of IGF-1R and IR-A. IGF-1 and IGF-2 also stimulated phosphorylation of Akt in RD-ES, TC-71, SK-ES-1, and SJSA-1 cells. MEDI-573 blocks this effect. However, in KHOS cells, although receptor phosphorylation was observed with IGF-1/-2 stimulation, there was no induction of Akt.

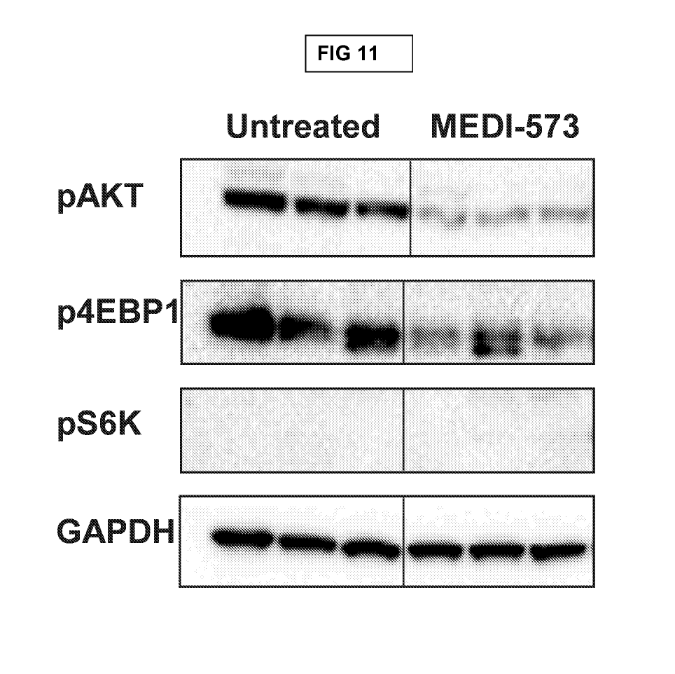

The in vivo effects of MEDI-573 on IGF signaling were also examined in sarcoma xenografts. To be consistent with in vitro experiments, in vivo pharmacodynamic studies were performed in two ways. First, the effect of MEDI-573 on signaling that was induced by IGF ligands, which were secreted by tumors in an autocrine manner, was examined. A single dose of MEDI-573 was given to mice bearing ˜400 mm³ RD-ES, SJSA-1, or KHOS/NP tumors. The administration of MEDI-573 inhibited autophosphorylation of pAKT and phosphorylated p4EBP1 in RD-ES tumors, but not in KHOS/NP tumors. An image of an immunoblot with samples from mice bearing RD-ES tumors is shown in FIG. 11.

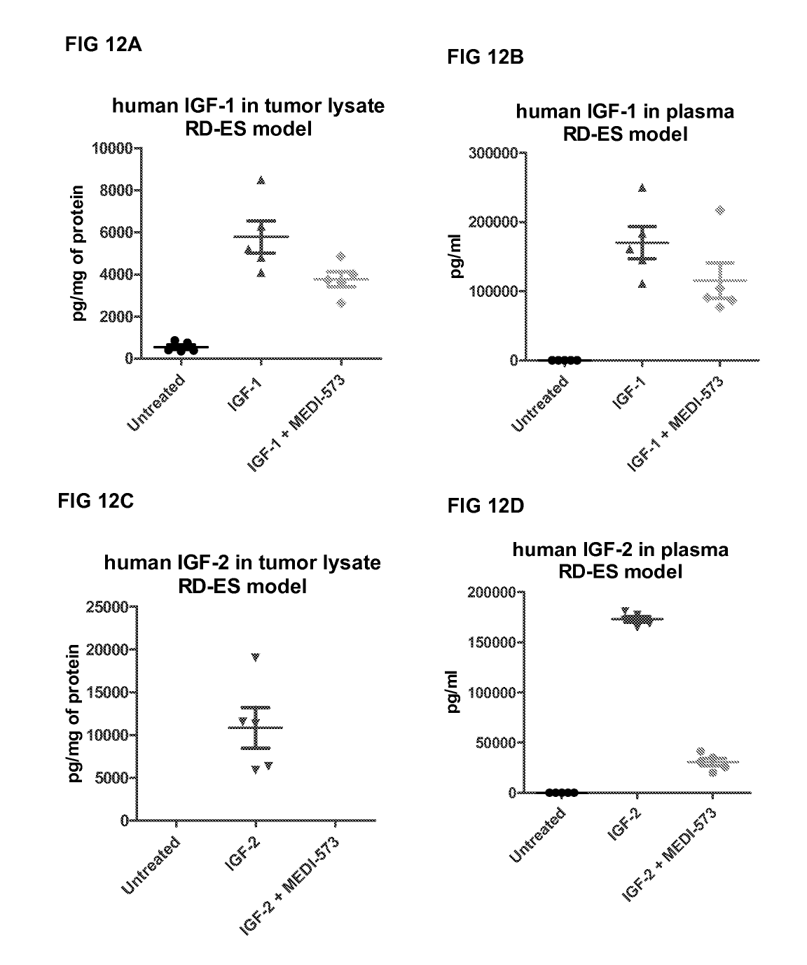

Adult mice do not produce murine IGF-2, and MEDI-573 has low binding affinity against murine IGF-1. Thus, human IGF-1 and IGF-2 (IGF-1/-2) were injected into mice in an attempt to understand the role of IGF ligands in driving tumor growth when delivered by endocrine or paracrine secretion, and the effect of MEDI-573 in inhibiting this function. Fifteen minutes after IGF-1 or IGF-2 injection, high levels of IGF-1 or IGF-2 were detected both in RD-ES tumor and plasma. Pretreatment with intraperitoneal MEDI-573 for 6 hours reduced IGF-1 levels by approximately 50% in tumor lysates and plasma (see FIG. 12A and FIG. 12B) and reduced the IGF-2 levels almost completely (see FIG. 12C and FIG. 12D).

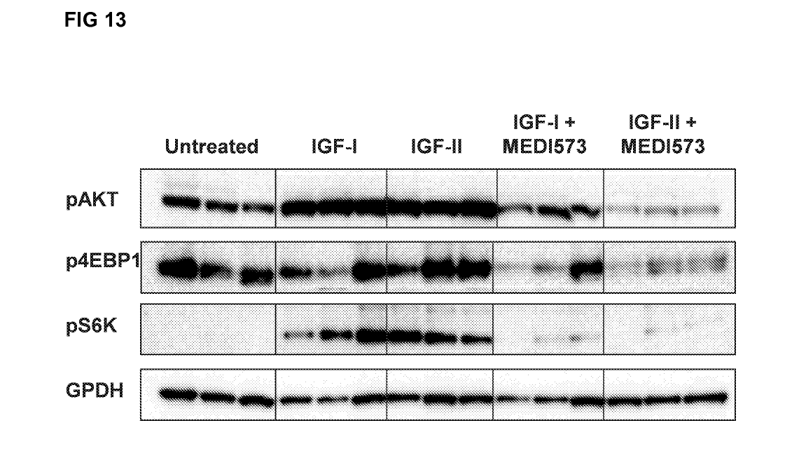

Similarly, phosphorylation of Akt and Ribosomal protein S6 kinase beta-1 (S6K) was increased compared to mice that did not receive IGF-1/-2 (FIG. 13). Pretreatment with MEDI-573 led to a dramatic reduction in IGF-induced pAKT and pS6K, particularly against IGF-2 injection. IGF-1/-2 injection did change the baseline level of p4EBP-1. MEDI-573 treatment inhibited p4EBP-1 even below the baseline level.

### Example 5

### MEDI-573 in Combination with mTORi Inhibited Sarcoma Cell Growth In Vitro

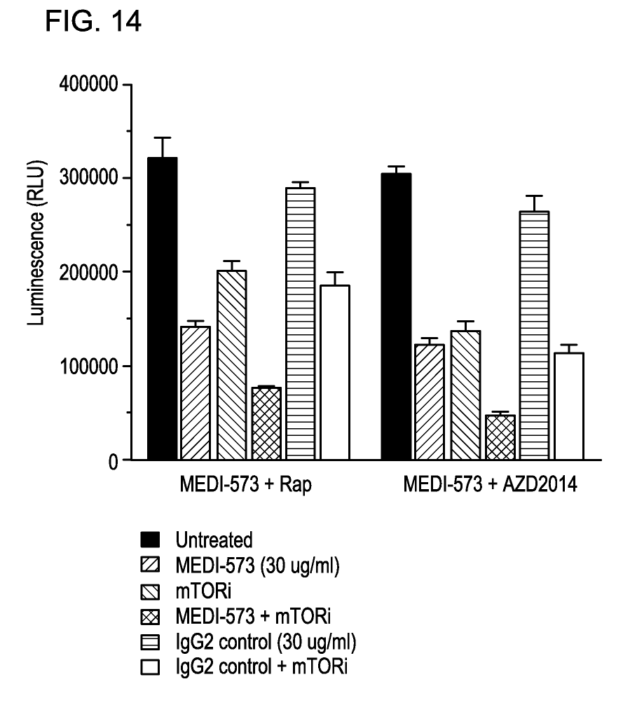

The effect of MEDI-573 in combination with the mTOR inhibitors rapamycin and AZD2014 was evaluated in cytotoxicity assays. RD-ES cells were treated with MEDI-573 and rapamycin, or MEDI-573 and AZD2014. As seen in FIG. 14, treatment with MEDI-573 alone led to a 56% decrease in cell viability, and treatment with and rapamycin alone led to a 34% decrease in cell viability. The combination of MEDI-573 with rapamycin resulted in an 80% reduction in viability (P<0.01). Treatment with the mTOR inhibitor AZD2014 alone reduced cell viability by 55%, and the combination of AZD2014 with MEDI-573 led to an 85% reduction in cell viability (P<0.01). Consistent with results depicted above, that showed that MEDI-573 had no effect on KHOS cell proliferation (Table 3), combination of MEDI-573 with either mTORi did not show any enhanced activity in KHOS cells either.

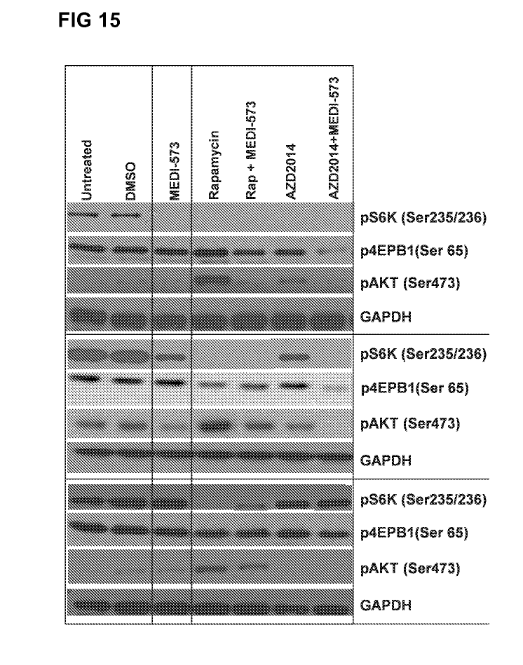

To examine the effect of MEDI-573, mTORi, and combination of both on IGF signaling, RD-ES, SJSA-1, and KHOS cells were treated with these agents for 4 hours. After separation of the cell lysates by gel electrophoresis, the proteins were detected by immunoblotting. FIG. 15 shows that MEDI-573 inhibited phosphorylation of S6K in RD-ES and SJSA-1 cells, but not in KHOS cells. Rapamycin alone and in combination with MEDI-573 completely inhibited pS6K in all 3 cell lines. MEDI-573 alone or rapamycin alone did not have effect on phosphorylation of 4EBP1. Combination treatment with both resulted in a decrease in p4EBP1 in the two responsive lines (RD-ES and TC-71), but did not have any effect in the non-responsive line (KHOS). Rapamycin treatment induced phosphorylation of AKT in all 3 cell lines. In the presence of MEDI-573, rapamycin-induced AKT activation was significantly inhibited to levels observed in untreated controls in RD-ES and TC-71 cells, but not in KHOS cells (FIG. 15).

While treatment with AZD2014 inhibited phosphorylation of pS6K in RD-ES, AZD2014 did not inhibit phosphorylation of pS6K in SJSA-1 or KHOS cells. Combination of MEDI-573 with AZD2014 inhibited phosphorylation of pS6K in SJSA-1 cells. The effect on pAKT phosphorylation appeared to be more pronounced when MEDI-573 was combined with AZD2014 than when MEDI-573 was combined with rapamycin (FIG. 15).

### Example 6

### MEDI-573 in Combination with mTORi Inhibits Sarcoma Cell Growth in RD-ES Tumor Xenografts

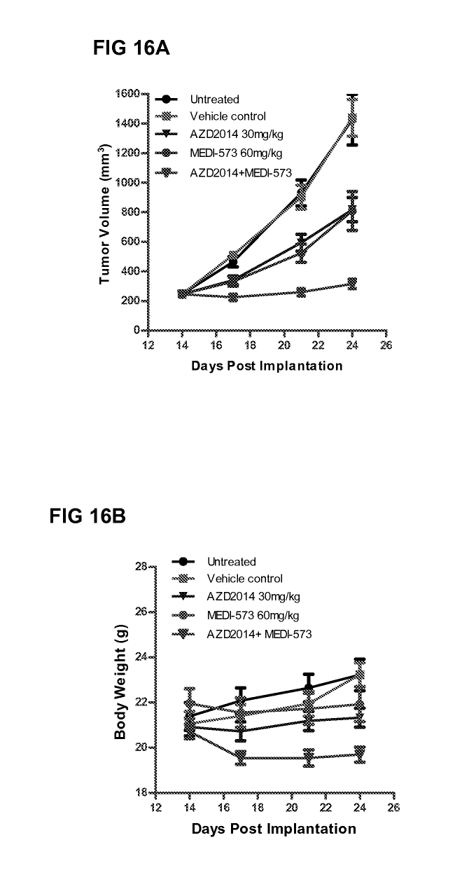

Treatment of the RD-ES xenograft model with MEDI-573 alone resulted in 52% tumor growth inhibition. Treatment of the RD-ES xenograft model with AZD2014 alone resulted in 51% tumor growth inhibition. Treatment of the RD-ES xenograft model with a combination of MEDI-573 and AZD2014 resulted in a 96% tumor growth inhibition which was significantly better than either agent alone (p<0.001) (FIG. 16A). The effects of the treatments on the body weight are shown in FIG. 16B. A similar effect on the tumor growth inhibition was observed in the SJSA-1 xenograft model. Treatment of the KHOS xenograft model with a combination of MEDI-573 and AZD2014 did not result in an increased tumor growth inhibition compared to treatment with the agents alone.

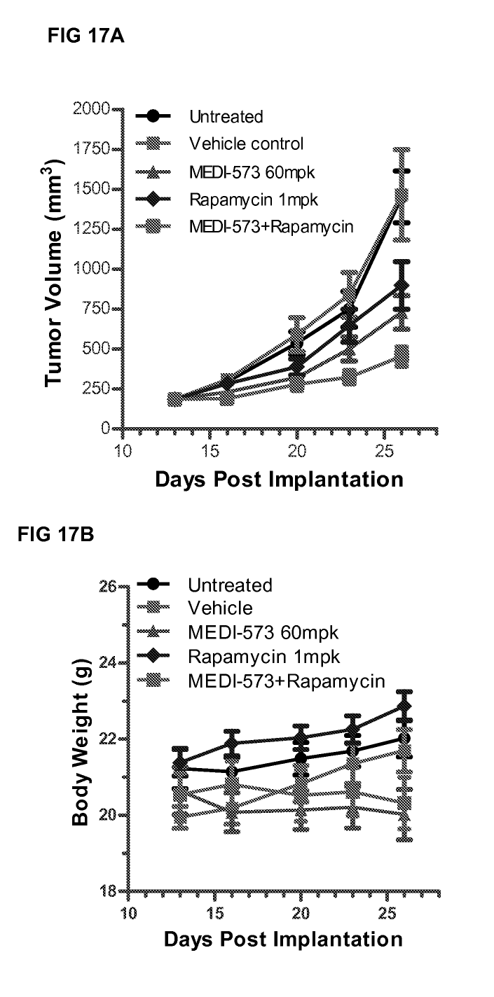

Combination of MEDI-573 with Rapamycin was also tested in the RD-ES xenograft model, and the results are shown in FIG. 17A. Although the effect of the combination of MEDI-573 with Rapamycin was slightly less than the combination of MEDI-573 with AZD2014, the combination treatment enhanced the anti-tumor activity (79% tumor growth inhibition) compared to either agent alone (59% for MEDI-573, and 44% for Rapamycin) (FIG. 17A). The combination treatments were tolerated as no significant body weight loss was observed (FIG. 17B).

The results described herein were obtained using the following materials and methods.

### Cells and Reagents

Sarcoma cell lines were purchased from American type Culture Collection (Manassas, Va.). CellTiter-Glo reagents were obtained from Promega (Madison, Wis.). Whole cell lysate kits for pIGF-1R, pIR-A, and pAKT were purchased from Meso Scale Discovery (MSD; Rockville, Md.). ELISA kits for total IGF-1 and IGF-1R were purchased from R&D Systems (Minneapolis, Minn.). ELISA kits for total IGF-2 were purchased from Insight Genomics (Falls Church, Va.). An ELISA kit for detecting free IGF-1 and IGF-2 was developed in house. Human IGF-1 and IGF-2 were obtained from R&D Systems (Minneapolis, Minn.). Antibodies for detecting phospho-AKT, phospho-4EBP1, phospho-S6K, and GAPDH were from Cell Signaling Technology (Beverly, Mass.).

RT-PCR Assays for Measuring IGF-1/-2, IGF-1R, IR-A, IR-B mRNA Levels

Total RNAs were purified using the ZR RNA MicroPrep Kit (Zymo Research, Irvine, Calif.) following manufacturer's protocol.

Single-stranded cDNA was generated from total RNA using the SuperScript® III First-Strand Synthesis SuperMix (Life Technologies, Carlsbad, Calif.). Samples of cDNA were pre-amplified using TaqMan Pre-Amp Master Mix, according to the manufacturer's instructions. Reactions contained 5 μL of cDNA, 10 μL of Pre-Amp Master Mix, and 5 μL of 0.2× gene expression assay mix (comprised of all primer/probes to be assayed) at a final reaction volume of 20 μL. Reactions were cycled with the recommended 14-cycle program and then diluted 1:5 with TE buffer. Pre-amplified cDNA was used immediately or stored at −20° C. until processed.

The reaction mix for preparing samples was loaded into 48×48 dynamic array chips and contained 2.5 μL of 2× Universal Master Mix, 0.25 μL of Sample Loading Buffer, and 2.25 μL of preamplified cDNA. The reaction mix for primer/probes contained 2.5 μL of 20× TaqMan Gene Expression Assay and 2.5 μL of Assay Loading Buffer. Prior to loading the samples and assay reagents into the inlets, the chip was primed in the IFC Controller. Samples (5 μL) were loaded into each sample inlet of the dynamic array chip, and 5 μL of 10× Gene Expression Assay Mix was loaded into each detector inlet. The chip was placed on the IFC Controller for loading and mixing. Upon completion of the IFC priming step, the chip was loaded on the BioMark RT-PCR System for thermal cycling (95° C. for 10 minutes, 40 cycles at 95° C. for 15 seconds, 60° C. for one minute). The number of replicates and the composition of the samples varied depending on the particular experiment but were never less than triplicate determinations. Average Cycle Threshold (Ct) values were used to quantify of the designed probes. The average Ct values of all available reference gene assays within a sample were utilized for calculation of ΔCt.

Levels of IGF-1, IGF-2, IGF-1R, IR-A and IR-B were tested. TaqMan Gene Expression assays of IR-A and IR-B have been described in Huang et al., 2011 (PLoS One. 2011; 6(10): e26177). This method allows the specific amplification of IR-A and IR-B independently of each other. Other TaqMan gene expression assays were purchased from Applied Biosystems.

### In Vitro Cell Proliferation Assays

Sarcoma cell lines were cultured overnight in regular growth medium. The following day, medium containing 0.1% charcoal stripped fetal bovine serum (FBS) was added and the cells incubated overnight. The next day, cells were treated with various amounts of MEDI-573 and the cultures incubated for 3 days. Proliferation was quantified using the CellTiter-Glo (CTG) reagent (Promega, Madison, Wis.).

To access the effect of MEDI-573 on IGF-Induced proliferation, MEDI-573 or isotype control, was added to the cells for 30 minutes at 37° C. IGF-1 or IGF-2 was then added to the appropriate wells and incubated for 3 days. Proliferation was quantified using the CTG reagent.

Assays for pIGF-1R, pIR-A, and pAKT

The sarcoma lines were cultured overnight in complete medium. The following day, medium containing 0.1% charcoal stripped fetal bovine serum (FBS) was added to the cultures and the cultures incubated overnight. The next day, cells were treated with various treatments for 5 minutes. Media was removed; cells were washed and lysed with 1.0% Triton X lysis buffer with protease and phosphatase inhibitors. Approximately 8-20 μg of total protein was loaded on MSD 96-Well MULTI-SPOT plates and the level of total and phosphorylated IGF-1R, IR-A and IRS-1 protein was determined using the Insulin Signaling Panel (total protein) and Insulin Signaling Panel (phosphoprotein) Whole Cell Lysate kits according to the manufacturers protocol. The level of total and phosphorylated AKT was determined using the Phospho (Ser473)/Total AKT Assay Whole Cell Lysate kit according to manufacturer's standard protocol.

### Xenograft Studies in Mice

For in vivo efficacy studies, five million sarcoma cells in 50% matrigel were inoculated subcutaneously into each female athymic nude mice. When tumors reach approximately 150-200 mm³, mice were randomly assigned into groups (10 mice per group). MEDI-573 was administrated intraperitoneally twice per week at indicated doses. The dose regimen for AZD2014 was oral once every day, for rapamycin was intraperitoneal injection every 3 days. Tumor volumes were measured twice weekly with calipers. Tumor growth inhibition was calculated on the last day of study relative to the initial and final mean tumor volume of the control group.

For in vivo mechanism of action (MOA) studies, when tumors reached approximately 400 mm³, a single dose of MEDI-573 was given. Tumor and plasma samples were collected 4 hr after dosing to assess the effect of MEDI-573 on autocrine IGF signaling. In another set of mice, 6 hr after MEDI-573 dosing, human IGF-1 or IGF-2 was injected by tail-vein. Tumor and plasma samples were collected 15 min after IGFs injection to assess the effect of MEDI-573 on IGF-1/-2 induced signaling.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents, publications, CAS, and accession numbers mentioned in this specification are herein incorporated by reference to the same extent as if each independent patent, publication, and accession number was specifically and individually indicated to be incorporated by reference.

What is claimed is:

1. A pharmaceutical composition for the treatment of sarcoma comprising an effective amount of an mTOR inhibitor and an effective amount of an antibody that specifically binds to at least one of insulin-like growth factor 1 (IGF-1) and insulin-like growth factor 2 (IGF-2).

2. The pharmaceutical composition of claim 1, wherein the antibody neutralizes a least one of IGF-1 and IGF-2.

3. The pharmaceutical composition of one of claim 1 or 2, wherein the antibody comprises:
a heavy chain complementarity determining region 1 (CDR1) comprising the amino acid sequence set forth in SEQ ID NO: 1 (Ser Tyr Asp Ile Asn);
a heavy chain complementarity determining region 2 (CDR2) comprising the amino acid sequence set forth in SEQ ID NO: 2 (Trp Met Asn Pro Asn Ser Gly Asn Thr Gly Tyr Ala Gln Lys Phe Gln Gly);
a heavy chain complementarity determining region 3 (CDR3) comprising the amino acid sequence set forth in SEQ ID NO: 3 (Asp Pro Tyr Tyr Tyr Tyr Tyr Gly Met Asp Val);
a light chain complementarity determining region 1 (CDR1) comprising the amino acid sequence set forth in SEQ ID NO: 4 (Ser Gly Ser Ser Ser Asn Ile Glu Asn Asn His Val Ser);
a light chain complementarity determining region 2 (CDR2) comprising the amino acid sequence set forth in SEQ ID NO: 5 (Asp Asn Asn Lys Arg Pro Ser); and
a light chain complementarity determining region 3 (CDR3) comprising the amino acid sequence set forth in SEQ ID NO: 6 (Glu Thr Trp Asp Thr Ser Leu Ser Ala Gly Arg Val).

4. The pharmaceutical composition of one of claim 1 or 2, wherein the antibody that specifically binds to at least one of IGF-1 and IGF-2 comprises one or more variable regions comprising an amino acid sequence selected from the amino acid sequences set forth in SEQ ID NO: 7 and SEQ ID NO: 8.

5. The pharmaceutical composition of any one of claims 1-4, wherein the antibody has the amino acid sequence of the antibody produced by hybridoma cell line 7.159.2 (ATCC Accession Number PTA-7424).

6. The pharmaceutical composition of any one of claims 1-5, wherein the mTOR inhibitor is selected from the group consisting of AZD2014, INK128, AZD8055, NVP-BEZ235, BGT226, SF1126, PKI-587, rapamycin, temsirolimus, everolimus, ridaforolimus, and combinations thereof.

7. The pharmaceutical composition of claim 6, wherein the mTOR inhibitor is rapamycin.

8. The pharmaceutical composition of claim 6, wherein the mTOR inhibitor is AZD2014.

9. The pharmaceutical composition of any one of claims 1-8, wherein the pharmaceutical composition is used for treating sarcoma selected from the group consisting of Ewing's sarcoma, Osteosarcoma, Rhabdomyo sarcoma, Askin's tumor, Sarcoma botryoides, Chondrosarcoma, Malignant Hemangioendothelioma, Malignant Schwannoma, soft tissue sarcoma, Alveolar soft part sarcoma, Angiosarcoma, Cystosarcoma Phyllodes, Dermatofibrosarcoma protuberans, Desmoid Tumor, Desmoplastic small round cell tumor, Epithelioid Sarcoma, Extraskeletal chondrosarcoma, Extraskeletal osteosarcoma, Fibrosarcoma, Hemangiopericytoma, Hemangiosarcoma, Kaposi's sarcoma, Leiomyosarcoma, Liposarcoma, Lymphangiosarcoma, Lymphosarcoma, Malignant peripheral nerve sheath tumor, Neurofibrosarcoma, Synovial sarcoma, and Undifferentiated pleomorphic sarcoma.

10. A method for reducing the survival or proliferation of a sarcoma cell, the method comprising:
contacting at least one sarcoma cell with a pharmaceutical composition comprising an mTOR inhibitor and an antibody that specifically binds at least one of IGF-1 and IGF-2;
wherein the survival or proliferation of the sarcoma cell is reduced.

11. A method for treating sarcoma in a subject, the method comprising administering to the subject a pharmaceutical composition comprising an mTOR inhibitor and an antibody that specifically binds at least one of IGF-1 and IGF-2.

12. The method of claim 11, wherein the antibody neutralizes at least one of IGF-1 and IGF-2.

13. The method of any one of claim 11 or 12, wherein the antibody comprises:
a heavy chain complementarity determining region 1 (CDR1) comprising the amino acid sequence set forth in SEQ ID NO: 1 (Ser Tyr Asp Ile Asn);
a heavy chain complementarity determining region 2 (CDR2) comprising the amino acid sequence set forth in SEQ ID NO: 2 (Trp Met Asn Pro Asn Ser Gly Asn Thr Gly Tyr Ala Gln Lys Phe Gln Gly);
a heavy chain complementarity determining region 3 (CDR3) comprising the amino acid sequence set forth in SEQ ID NO: 3 (Asp Pro Tyr Tyr Tyr Tyr Tyr Gly Met Asp Val);
a light chain complementarity determining region 1 (CDR1) comprising the amino acid sequence set forth in SEQ ID NO: 4 (Ser Gly Ser Ser Ser Asn Ile Glu Asn Asn His Val Ser);
a light chain complementarity determining region 2 (CDR2) comprising the amino acid sequence set forth in SEQ ID NO: 5 (Asp Asn Asn Lys Arg Pro Ser); and
a light chain complementarity determining region 3 (CDR3) comprising the amino acid sequence set forth in SEQ ID NO: 6 (Glu Thr Trp Asp Thr Ser Leu Ser Ala Gly Arg Val).

14. The method of any one of claims 11-13, wherein the antibody that specifically binds at least one of IGF-1 and IGF-2 comprises one or more variable regions comprising the amino acid sequence selected from the amino acid sequences set forth in SEQ ID NO: 7 and SEQ ID NO: 8.

15. The method of any one of claims 11-14, wherein the mTOR inhibitor is at least one of AZD2014, INK128, AZD8055, NVP-BEZ235, BGT226, SF1126, PKI-587, rapamycin, temsirolimus, everolimus, and ridaforolimus.

16. The method of any one of claims 11-15, wherein the sarcoma is one or more of Ewing's sarcoma, Osteosarcoma, Rhabdomyosarcoma, Askin's tumor, Sarcoma botryoides, Chondrosarcoma, Malignant Hemangioendothelioma, Malignant Schwannoma, soft tissue sarcoma, Alveolar soft part sarcoma, Angiosarcoma, Cystosarcoma Phyllodes, Dermatofibrosarcoma protuberans, Desmoid Tumor, Desmoplastic small round cell tumor, Epithelioid Sarcoma, Extraskeletal chondrosarcoma, Extraskeletal osteosarcoma, Fibrosarcoma, Hemangiopericytoma, Hemangiosarcoma, Kaposi's sarcoma, Leiomyosarcoma, Liposarcoma, Lymphangiosarcoma, Lymphosarcoma, Malignant peripheral nerve sheath tumor, Neurofibrosarcoma, Synovial sarcoma, and Undifferentiated pleomorphic sarcoma.

17. The method of any one of claims 11-16, wherein the pharmaceutical composition is administered at 10 mg/kg, 30 mg/kg, or 60 mg/kg.

18. The method of any one of claims 11-17, wherein the method inhibits tumor growth in the subject by at least about 10%, 25%, 50%, 75% or more relative to a reference.

19. The method of any one of claims 11-18, wherein the method inhibits sarcoma cell proliferation.

20. The method of any one of claims 11-19, wherein the administering is by intravenous injection or oral administration.

21. The method of any one of claims 11-16, wherein the antibody and the mTOR inhibitor are administered concurrently, within about 1 hour to about 24 hours, or within about 1 day to about 3 days.

22. A method for treating a subject having Ewing's sarcoma, osteosarcoma, or rhabdomyosarcoma, the method comprising administering to the subject an effective amount of an antibody and rapamycin, thereby treating the Ewing's sarcoma, osteosarcoma, or rhabdomyosarcoma in the subject; wherein the antibody comprises
a heavy chain complementarity determining region 1 (CDR1) comprising the amino acid sequence set forth in SEQ ID NO: 1 (Ser Tyr Asp Ile Asn);
a heavy chain complementarity determining region 2 (CDR2) comprising the amino acid sequence set forth in SEQ ID NO: 2 (Trp Met Asn Pro Asn Ser Gly Asn Thr Gly Tyr Ala Gln Lys Phe Gln Gly);
a heavy chain complementarity determining region 3 (CDR3) comprising the amino acid sequence set forth in SEQ ID NO: 3 (Asp Pro Tyr Tyr Tyr Tyr Tyr Gly Met Asp Val);
a light chain complementarity determining region 1 (CDR1) comprising the amino acid sequence set forth in SEQ ID NO: 4 (Ser Gly Ser Ser Ser Asn Ile Glu Asn Asn His Val Ser);
a light chain complementarity determining region 2 (CDR2) comprising the amino acid sequence set forth in SEQ ID NO: 5 (Asp Asn Asn Lys Arg Pro Ser); and
a light chain complementarity determining region 3 (CDR3) comprising the amino acid sequence set forth in SEQ ID NO: 6 (Glu Thr Trp Asp Thr Ser Leu Ser Ala Gly Arg Val).

23. A method for treating a subject having Ewing's sarcoma, osteosarcoma, or rhabdomyosarcoma, the method comprising administering to the subject an effective amount of an antibody and AZD2014, thereby treating the Ewing's sarcoma, osteosarcoma, or rhabdomyosarcoma in the subject; wherein the antibody comprises
a heavy chain complementarity determining region 1 (CDR1) comprising the amino acid sequence set forth in SEQ ID NO: 1 (Ser Tyr Asp Ile Asn);
a heavy chain complementarity determining region 2 (CDR2) comprising the amino acid sequence set forth in SEQ ID NO: 2 (Trp Met Asn Pro Asn Ser Gly Asn Thr Gly Tyr Ala Gln Lys Phe Gln Gly);
a heavy chain complementarity determining region 3 (CDR3) comprising the amino acid sequence set forth in SEQ ID NO: 3 (Asp Pro Tyr Tyr Tyr Tyr Tyr Gly Met Asp Val);
a light chain complementarity determining region 1 (CDR1) comprising the amino acid sequence set forth in SEQ ID NO: 4 (Ser Gly Ser Ser Ser Asn Ile Glu Asn Asn His Val Ser);
a light chain complementarity determining region 2 (CDR2) comprising the amino acid sequence set forth in SEQ ID NO: 5 (Asp Asn Asn Lys Arg Pro Ser); and
a light chain complementarity determining region 3 (CDR3) comprising the amino acid sequence set forth in SEQ ID NO: 6 (Glu Thr Trp Asp Thr Ser Leu Ser Ala Gly Arg Val).

24. A kit for treating sarcoma comprising an effective amount of an mTOR inhibitor and an antibody that specifically binds IGF-1 and/or IGF-2, and instructions for using the kit to treat sarcoma.

25. The kit of claim 24, wherein the mTOR inhibitor is rapamycin or AZD2014 and the antibody comprises
a heavy chain complementarity determining region 1 (CDR1) comprising the amino acid sequence set forth in SEQ ID NO: 1 (Ser Tyr Asp Ile Asn);
a heavy chain complementarity determining region 2 (CDR2) comprising the amino acid sequence set forth in SEQ ID NO: 2 (Trp Met Asn Pro Asn Ser Gly Asn Thr Gly Tyr Ala Gln Lys Phe Gln Gly);
a heavy chain complementarity determining region 3 (CDR3) comprising the amino acid sequence set forth in SEQ ID NO: 3 (Asp Pro Tyr Tyr Tyr Tyr Tyr Gly Met Asp Val);
a light chain complementarity determining region 1 (CDR1) comprising the amino acid sequence set forth in SEQ ID NO: 4 (Ser Gly Ser Ser Ser Asn Ile Glu Asn Asn His Val Ser);
a light chain complementarity determining region 2 (CDR2) comprising the amino acid sequence set forth in SEQ ID NO: 5 (Asp Asn Asn Lys Arg Pro Ser); and
a light chain complementarity determining region 3 (CDR3) comprising the amino acid sequence set forth in SEQ ID NO: 6 (Glu Thr Trp Asp Thr Ser Leu Ser Ala Gly Arg Val).


SEQUENCE LISTING (10 sequences)

SEQ ID NO: 1 - protein, 5 aa, Artificial Sequence
  Description of Artificial Sequence Synthetic peptide
SYDIN

SEQ ID NO: 2 - protein, 17 aa, Artificial Sequence
  Description of Artificial Sequence Synthetic peptide
WMNPNSGNTGYAQKFQG

SEQ ID NO: 3 - protein, 11 aa, Artificial Sequence
  Description of Artificial Sequence Synthetic peptide
DPYYYYYGMDV

SEQ ID NO: 4 - protein, 13 aa, Artificial Sequence
  Description of Artificial Sequence Synthetic peptide
SGSSSNIENNHVS

SEQ ID NO: 5 - protein, 7 aa, Artificial Sequence
  Description of Artificial Sequence Synthetic peptide
DNNKRPS

SEQ ID NO: 6 - protein, 12 aa, Artificial Sequence
  Description of Artificial Sequence Synthetic peptide
ETWDTSLSAGRV

SEQ ID NO: 7 - protein, 121 aa, Artificial Sequence
  Description of Artificial Sequence Synthetic polypeptide
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSA

SEQ ID NO: 8 - protein, 112 aa, Artificial Sequence
  Description of Artificial Sequence Synthetic polypeptide
QSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLG

SEQ ID NO: 9 - protein, 217 aa, Artificial Sequence
  Description of Artificial Sequence Synthetic polypeptide
QSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 446 aa, Artificial Sequence
  Description of Artificial Sequence Synthetic polypeptide
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK